(19) United States
(12) Patent Application Publication
(10) Pub. No.: US 2017/0100429 A1
(43) Pub. Date: Apr. 13, 2017

(54) Methods of Treating Diarrhea in Neonatal and Young Non-Human Animals

(71) Applicant: Jaguar Animal Health, Inc., San Francisco, CA (US)

(72) Inventor: Serge Martinod, Groton, CT (US)

(21) Appl. No.: 15/314,165

(22) PCT Filed: May 28, 2015
(86) PCT No.: PCT/US2015/032899

§ 371 Date: Nov. 28, 2016

Related U.S. Application Data
(60) Provisional application No. 62/087,203, filed on Dec. 3, 2014.
(60) Provisional application No. 62/004,641, filed on May 29, 2014.

(51) Int. Cl. A61K 31/765; A61K 9/00; A23K 20/10; A61K 9/20; A61K 35/741; A61K 36/47; A61K 9/06

(52) U.S. Cl. CPC A61K 31/765; A61K 36/47; A61K 9/0056; A61K 9/06; A61K 9/0053; A61K 9/20; A61K 35/741; A23K 20/10; A61K 2035/115

(57) ABSTRACT

Methods of treating neonatal and young non-human animals suffering from diarrhea, and/or symptoms thereof, by administering to an animal in need thereof a proanthocyanidin polymer composition isolated from a Croton spp. or a Calophyllum spp. are provided. In particular, the neonatal and young animals include calves, young equines and young camels, which frequently suffer from diarrhea of various etiologies, and the administered proanthocyanidin polymer composition is isolated from Croton lechleri. Further provided are methods of improving weight gain and/or reducing mortality in neonatal non-human animals by administration of the proanthocyanidin polymer composition. The composition, either enteric or non-enteric, can be in aqueous soluble form and orally administered to the affected neonatal and young animals.

### FIELD OF THE INVENTION

The invention relates to the treatment of diarrhea in neonatal, unweaned and young non-human animals with a composition comprising a proanthocyandin polymer isolated from the plant Croton spp. or Calophyllum spp., or with a latex, extract, or food supplement derived therefrom. More particularly, the composition is effective in treating secretory diarrhea of various etiologies and reducing the severity and duration of diarrhea in neonatal and young, non-human animals. The proanthocyanidin polymer compositions can be administered to neonatal non-human animals to improve weight gain and survivability.

### BACKGROUND OF THE INVENTION

Infectious diseases are among the most widespread problems of neonatal and young animals, such as calves of different animal species. Diarrhea, called “scours” in calves, frequently occurs within the first several days of life and is an important cause of calf sickness and death in the United States. Dehydration from diarrhea in neonatal and young farm or larger-sized animals results in measurable morbidity and mortality in many millions of animals worldwide.

A wide array of infectious and pathogenic agents including bacteria, viruses, and parasites cause diarrhea in animals, particularly, domesticated livestock animals associated with farming, food, and labor. Many of these enteropathogens cause one or more adverse effects in the animals, such as severe intestinal lesions, dehydration, alterations in enzyme activity, and/or alterations in nutrient transport mechanisms. The clinical presentation of diarrhea caused by these agents may vary; some diarrheas are self-limiting, while others are associated with high morbidity or high mortality (R. E. Holland, 1990, Clin. Microbiol. Rev., 3(4):345-375).

Infectious diarrhea of neonatal animals is an extremely common and economically devastating condition confronted by the animal agriculture and animal husbandry industries. When encountered in a herd, acute infectious diarrhea is often difficult to manage, contain and cure, because of the large numbers of potential enteropathogens involved, the differences in natural immunity among animals within the herd, environmental conditions and stresses, nutritional factors, the dynamics of the animal population, management conditions, and a difficulty in determining an etiological diagnosis. As a consequence, such a diagnosis is frequently not established for a large percentage of cases of neonatal animal diarrheas. In addition, neonatal and young animals, such as calves, may be predisposed to diarrheas as a result of difficult births, exposure, poor maternal nutrition and/or health, poor mothering capabilities on the part of the dam, or a combination of these factors. When some or all of these conditions occur, the resistance of the calf to infectious diseases, for example, is lowered, and exposure to and invasion by infectious agents play pivotal roles in producing diarrhea.

Some of the most common types of infectious agents resulting in neonatal animal diarrhea, particularly in calves, include E. coli, e.g., E. coli K-99; rotavirus and coronavirus, cryptosporidia, Salmonella spp., Campylobacter jejuni. In some cases, such as infection by Salmonella, which is a human pathogen, human handlers of the animals and those who treat the animals, may also be at risk of infection and disease.

Diarrhea in neonatal and young animals can also be due to noninfectious causes, such as changes to a feeding program, energy deficiencies and vitamin shortages related to pregnant adult females that can extend to the newborn offspring, causing weakness and susceptibility to infection. Environmental and sanitation conditions associated with the birth of newborn animals can also be associated with outbreaks of disease and resulting diarrhea. For example, an unclean environment, e.g., an accumulation of urine and manure in an area where animals are born and nursed, can lead to disease syndromes that are characterized by diarrhea. In addition, problems related to giving birth by adult females, such as difficult calving and insufficient colostrum, can lead to weak newborns and a lack of passive immunity provided by the colostrum. Thus, adverse conditions affecting both the mothers and their newly and recently born offspring can lead to outbreaks of diarrhea requiring treatment of the neonatal and young animals. Noninfectious diarrhea, while oftentimes not severe enough to cause death, can weaken the young animal and make it more susceptible to infectious diarrhea, which contributes to a neonatal and young animal's inability to survive.

The available and commonly used treatments for diarrhea in neonatal and young non-human animals typically involve vital fluid replacement and electrolyte replenishment to counter or stop fluid and electrolyte loss. Other types of treatments include the administration of gut-lining protectants, e.g., bismuth, oral antibiotics, and agents that affect gut motility. Depending on the cause(s), timing, severity and course of diarrhea and/or its associated disease or condition, the various known treatments may or may not be effective, and the animals may or may not respond adequately. Because the economic and humane impacts of diarrhea and its related conditions on the afflicted animals, their handlers and caregivers are so great, there is a compelling need for alternative, safe, and medically effective, as well as cost effective, treatments and remedies. The present invention addresses such a need.

### SUMMARY OF THE INVENTION

The present invention relates to methods of treating diarrhea in neonatal, young, or non-adult animals in need thereof by administering a polymeric proanthocyanidin, i.e., a proanthocyanidin polymer, from a Croton species or Calophyllum species. In an embodiment, a pharmaceutically or physiologically acceptable formulation or composition comprising a proanthocyanidin polymer from a Croton species or Calophyllum species is administered. In particular embodiments, a proanthocyanidin polymer from Croton lechleri, or pharmaceutically acceptable formulation or composition comprising a proanthocyanidin polymer from Croton lechleri is administered.

In an embodiment, the proanthocyanidin polymer composition is a latex or extract from a Croton species or Calophyllum species, in particular, Croton lechleri. In another embodiment, the composition is a botanical extract of Croton lechleri containing a proanthocyanidin oligomer, or a food supplement formulation of the botanical extract of Croton lechleri. Such Croton species or Calophyllum species latex or extract compositions can be more highly purified as described herein. In an embodiment, the methods involve the administration of a pharmaceutically acceptable composition comprising a proanthocyanidin polymer from Croton lechleri to a non-human animal in need thereof. In an embodiment, the methods involve the administration of a proanthocyanidin polymer from Croton lechleri, or a pharmaceutically acceptable composition comprising a proanthocyanidin polymer from Croton lechleri, wherein the proanthocyanidin polymer or oligomer from C. lechleri is also known as crofelemer (a purified proanthocyanidin oligomer), SP 303, or SB 300, as further described herein. In certain embodiments, the C. lechleri proanthocyanidin polymer, or composition thereof, is in an enteric coated form that protects the proanthocyanidin polymer from the stomach environment of the non-human animal. In other embodiments, the C. lechleri proanthocyanidin polymer, or composition thereof, is in a non-enteric coated form.

The invention provides a method of treating and preventing the debilitating effects of diarrhea in neonatal and young non-human animals. In particular, the methods treat and prevent dehydration associated with water, fluid and electrolyte losses in animals afflicted with diarrhea. The methods of the invention further prevent or reduce the incidence of intestinal lesions, weakness and death in the neonatal and young non-human animals. In other embodiments, the methods treat and prevent diarrhea associated with colitis, including acute colitis, in afflicted animals. Thus, in an embodiment, the methods of the invention provide antisecretory treatments for diarrhea, particularly, secretory or watery diarrhea, in neonatal and young non-human animals.

The invention is more particularly directed to a method of improving gut health and controlling diarrhea in neonatal bovine or camel calves in need thereof by administering a proanthocyanidin polymer from Croton lechleri in an effective amount to control or treat the diarrhea in these animals. In an embodiment the proanthocyanidin polymer is a formulation, composition, or extract from Croton lechleri. In an embodiment, the proanthocyanidin polymer from Croton lechleri is a more highly purified composition containing proanthocyanidin polymer or oligomer, such as crofelemer or SB 300 compositions described herein.

The invention is also more particularly directed to a method of improving gut health, controlling diarrhea and normalizing stool formation in neonatal or young horses (foals) in need thereof by administering a proanthocyanidin polymer from Croton lechleri in an effective amount to control or treat the diarrhea in these animals. In an embodiment the proanthocyanidin polymer is a formulation, composition, or botanical extract from Croton lechleri. In an embodiment, the formulation, composition, or botanical extract from Croton lechleri is in the form of a paste or gel. In a particular embodiment, the paste formulation comprises beads (nano or microparticles) comprising enterically coated SB 300 or SP 303 and is orally administered to foals in need. In an embodiment, the paste formulation comprises beads (nano or microparticles) comprising enterically coated SB 300. In a particular embodiment, the paste comprising SB 300 enteric beads is orally administered to a foal twice daily for three days. In some embodiments, the paste is orally administered for three consecutive days. In an embodiment, the paste comprising SB 300 enteric beads is orally administered to a foal in need at a dose of 2 mg/kg twice daily for three days.

In an aspect, the invention provides a method of treating a neonatal or young non-human animal having diarrhea associated with enteropathogenic infection, the method comprising orally administering to an animal in need thereof a pharmaceutically acceptable composition comprising an aqueous soluble proanthocyanidin polymer from Croton lechleri, wherein the composition is formulated as a bolus or as a reconstituted powder and administered to the animal in an amount of at least 40 mg to 300 mg for consecutive days greater than one day, thereby treating the diarrhea in the neonatal or young animal. In various embodiments of the method, the neonatal or young animal is selected from a bovine calf, a camel calf, a buffalo calf, a bison calf, a lamb, a kid, a foal, or a piglet. In particular embodiments, the neonatal or young animal is a bovine calf or a camel calf. In other embodiments, the neonatal or young animal is an equine foal. In an embodiment, the proanthocyanidin polymer composition is administered twice daily for three consecutive days. In certain embodiments, the diarrhea is secretory or watery diarrhea associated with enteropathogen infection of the animal with one or more of E. coli, rotavirus, or coronavirus. In some embodiments, the diarrhea is episodic. In some embodiments, the animal is additionally infected with Salmonella spp. and/or Cryptosporidia. In embodiments of the method, the proanthocyanidin polymer composition or botanical extract derived from C. lechleri is administered as a powder reconstituted with oral electrolytes, milk or a milk substitute, physiological saline, or water; or as a bolus; or as a paste or gel; or in animal feed. The treated animals, such as calves or foals, can be less than two weeks of age, or two to four weeks of age. In embodiments of the method, the composition is administered to the animal in an amount of at least 30 mg to 350 mg, or in amount of 40 mg, 50 mg, or 250 mg. In other embodiments, the neonatal or young animal is approximately 30 to 50 kg in weight; is a lamb, a kid of approximately 2 to 8 kg in weight, a bovine calf of approximately 30 to 40 kg in weight, or a camel calf of approximately 40 to 50 kg in weight. In a particular embodiment, the proanthocyanidin polymer composition or botanical extract derived from C. lechleri is administered in a paste formulation at a dose of 2 mg/kg, where the approximate body weight of a foal under one year of age is 60 pounds (lb.). In embodiments, the proanthocyanidin polymer is administered as an enteric coated pharmaceutical composition or as a non-enteric coated pharmaceutical composition. In addition, the proanthocyanidin polymer can be SB 300, SP 303, crofelemer and pharmaceutically acceptable compositions thereof.

In another of its aspects, the invention provides a method of treating a neonatal or unweaned equine animal for diarrhea associated with enteropathogenic infection, the method comprising orally administering to the animal a pharmaceutically acceptable composition comprising an aqueous soluble proanthocyanidin polymer from Croton lechleri, wherein the composition is provided in a form selected from a bolus, a reconstituted powder, or a gel or paste, and is administered to the animal in an amount of at least 100 mg for consecutive days greater than one day, thereby treating the diarrhea in the neonatal or unweaned equine animal. In an embodiment, the animal is infected with bacteria, viruses and protozoa, in which the infection induced the diarrhea. In various embodiments, the proanthocyanidin polymer composition is administered to the animal in an amount of at least 250 mg and optionally can be in the form of a gel contained in a delivery device, which can be a syringe. In an embodiment, the gel or paste comprises polymeric microparticles or nanoparticles containing the composition, and the polymeric microparticles or nanoparticles are optionally pH-sensitive. In embodiments, the animal is less than two weeks of age and/or is approximately 30 to 50 kg in weight. In embodiments of the method, the proanthocyanidin polymer is administered as an enteric coated or as a non-enteric coated pharmaceutical composition. In addition, the proanthocyanidin polymer can be SB 300, SP 303, crofelemer and pharmaceutically acceptable compositions thereof. In a particular embodiment, the proanthocyanidin polymer is enterically protected beads, including enteric beads including SB 300 or SP 303.

In another aspect, the methods of the present invention provide prophylactic or preventative treatment of neonatal and young animals against the debilitating effects of diarrheal disease and its associated symptoms, e.g., dehydration and weight loss. In accordance with the invention, a C. lechleri proanthocyanidin polymer composition can be administered to neonatal and young animals at a suitable time after birth to protect the animals from diarrhea outbreaks typically caused by infections and environmental conditions. Administering a C. lechleri proanthocyanidin polymer composition to neonatal and young animals can also serve to ameliorate or reduce the risk of the animals' suffering from a more serious or severe form of disease relative to animals that are not provided with the C. lechleri proanthocyanidin polymer composition. In certain embodiments, administration of the C. lechleri proanthocyanidin polymer composition to neonatal animals within 1, 2, 3 or 4 days after birth for a period of 1, 2, 3, 4 or more days can increase weight gain and/or improve survivability in a population of animals, including in bovines, camels, buffalo, bisons, lambs, goats, horses and pigs. The C. lechleri proanthocyanidin polymer composition can be enteric or non-enteric and can be SB 300 or SP 303. The dose and regimen of C. lechleri proanthocyanidin polymer composition administration are within the skill of the practitioner and will depend on the environmental conditions of the animals to be treated. In nonlimiting embodiments, it is envisioned that the animals can be prophylactically treated just after birth, e.g., days one to four, for from one to five days, or fewer, as necessary or desired.

In another of its aspects, the present invention provides the surprising result that treatment of non-human young animals with a Croton lechleri proanthocyanidin polymer composition, such as a C. lechleri botanical extract product, according to the invention provides one or more beneficial effects, for example, lower dehydration and higher fecal dry matter content, in treated animals that endures beyond the time period of actual administration of the treatment product to the animals, i.e., after cessation of the administration of the product to the animal. This unexpected carryover effect demonstrates that the administration of a C. lechleri proanthocyanidin polymer composition or C. lechleri botanical extract product, particularly an early administration to the young animal, followed by a period in which the product is not administered to the animal, may induce beneficial changes in the intestine of treated animals that is maintained beyond the actual course of the therapy. By way of example, the C. lechleri proanthocyanidin polymer composition or C. lechleri botanical extract product administered to a calf at or within 12 hours of a challenge with a diarrhea inducing pathogen, with additional doses administered every 12 hours until 72 hours (6 total doses), affords a long term protection for many days, e.g., more than 10 to 14 days, following the last administration. Accordingly, an effect of early administration of C. lechleri proanthocyanidin polymer or C. lechleri botanical extract product, may endure for a time period greater than about one, two, three, four, or more weeks after treatment of the animal has ceased. In an aspect, early administration includes treating animals in need at the first sign of scours (diarrhea). In a particular aspect, the carryover effect may last for two to three weeks after cessation of treatment of the animal, thus allowing the animal to regain and maintain a healthy, normal gastrointestinal condition and function.

Accordingly, the methods of the invention provide for periodic administration of the C. lechleri proanthocyanidin polymer or C. lechleri botanical extract product to an animal, such that an initial treatment may be given, followed by a time period, e.g., a lag of several days or even weeks, such as 1, 2, 3, 4, or more weeks, before another treatment, if any, is given.

The disclosed methods and C. lechleri-derived proanthocyanidin polymer and botanical extract products used in the methods provide several advantages in the treatment of diarrhea (scours) in neonatal, pre-weaned non-human animals, e.g., bovine calves. Such advantages include reduced medication and labor and veterinary costs, which result in earlier weaning of animals and heavier weaning weights. In addition, the treatment of young animals in accordance with the methods and products of the invention may also reduce the quantity of electrolytes used in standard of care to treat diarrhea-related dehydration and other symptoms, which is also of economic and commercial benefit.

### BRIEF DESCRIPTION OF THE FIGURES

FIG. 1 shows the effect of a treatment method involving the administration of proanthocyanidin polymer from Croton lechleri according to the invention on average fecal score number as described in Example 2 herein. Calves were scored twice daily for three days using a 3 level scoring system. As observed, the C. lechleri proanthocyanidin polymer (crofelemer)-treated calves demonstrated faster improvement on diarrhea scores starting on the second day after treatment. In FIG. 1, the average fecal score is shown on the y-axis and the number of treatment days is shown on the x-axis.

FIG. 2 shows results from a calf study in which young calves experiencing diarrhea and illness caused by enterogenic bacterial infection were treated with the C. lechleri proanthocyanidin polymer SB 300 in either enteric or reconstituted powder form, or with placebo. The graph presents fecal score (fecal consistency rating) versus day of treatment, as described in Example 3.

FIG. 3 presents results from the calf study of Example 3 in which young calves experiencing diarrhea and illness caused by enterogenic bacterial infection were treated with the C. lechleri proanthocyanidin polymer SB 300 in either enteric or restituted powder form, or with placebo. The percent of calves with watery diarrhea versus day of treatment is shown. A reduction in calf morbidity can be observed in the animals treated with C. lechleri proanthocyanidin polymer SB 300, in particular, the enteric form.

FIG. 4 presents results from the study of Example 3 and shows the effect of treatment on calves' fecal scores measured twice daily from pre-challenge (sample 1) to day 10 (sample 20). The bottom line on the graph (blue) represents calves in the group that received a bolus of enteric coated C. lechleri botanical extract containing proanthocyanidin (ECROF); the top line on the graph (red) represents calves in the control group (CTR) that received a placebo bolus. Error bars represent the standard error of the mean. The treatment period is represented by the horizontal bar (orange) in the lower left portion of the graph.

FIG. 5 presents results from the study of Example 3 and shows the effect of treatment on calves' fecal scores based on fecal consistency measured on fecal samples collected twice daily, averaged and summarized in the following categories: pre-challenge (first examination, baseline); during treatment (2^{nd} to 7^{th} examination); and after treatment (8^{th} to 24^{th} examination). Error bars represent the standard error of the mean. The right-most bars (blue) in each portion of the graph represent ECROF treated animals; the left-most bars (red) in each portion of the graph represent CTR animals. The different letters within the time frames depicted represent statistical differences (P-value<0.05).

FIG. 6 presents results from the study of Example 3 and shows the effects of treatment on calves' fecal dry matter content (percentage of dry matter) measured on fecal samples collected twice daily from pre-challenge (sample 1) to day 5 (sample 10). The top line (blue) represents the results from calves in the in the group that received a bolus of enteric coated C. lechleri botanical extract containing proanthocyanidin (ECROF); the bottom line on the graph (red) represents calves in the control group (CTR) that received a placebo bolus. Error bars represent the standard error of the mean.

FIG. 7 presents results from the study of Example 3 and shows dehydration scores obtained by scoring calves' skin turgor and eyes recession. Error bars represent the standard error of the mean. The right-most bars (blue) in each portion of the graph represent ECROF treated animals; the left-most bars (red) in each portion of the graph represent CTR animals. The different letters within the time frames depicted represent statistical differences (P-value<0.05).

### DESCRIPTION OF THE INVENTION

The invention provides treatment methods effective for reducing and/or alleviating diarrhea in neonatal, unweaned non-human animals in need thereof. In particular, the methods are directed to the treatment of diarrhea, particularly secretory/watery diarrhea, or episodic diarrhea, caused by a variety of etiological agents and/or environmental factors in neonatal and young (juvenile, non-adult) animals, particularly where scourges of diarrhea in such immature animals can have a profound economic impact for the animal agriculture, food and health industries. The invention further provides formulations and compositions suitable for treating diarrhea in neonatal and young animals. Unless otherwise noted herein, use of the term “animal” herein denotes non-human, warm-blooded mammals of a number of different species. In addition, the terms “young”, non-adult”, “immature” and “juvenile” are used synonymously herein and generally refer to animals under one year of age.

The methods of the invention provide a solution to a significant need for the animal industry, e.g., the beef and dairy industries worldwide, in which neonatal calf diarrhea presents one of the largest health challenges, as well as economic losses. In addition, the methods of the invention provide a solution to the common problem of watery diarrhea, including episodic diarrhea, in horse foals. The methods and treatments of the invention improve gastrointestinal/gut health and normalize stool formation in young animals suffering from diarrheal conditions, including, by way of example, watery diarrhea in horse foals and bovine calves.

The methods and treatments of the invention are particularly suitable for treating animals of a young age. In an embodiment, the animals are neonatal (or newborn), unweaned, non-adult animals that are born, bred, raised and/or maintained in a domesticated and/or agricultural setting, e.g., as livestock and farm animals, for commodities such as food, labor, sport, or other commercial or non-commercial agricultural husbandry capacity. Nonlimiting examples of animals affected by diarrhea and treatable by the methods and formulations of the invention include, without limitation, neonatal and young cattle (calves), young bison or buffalo, pigs (piglets), sheep (lambs), goats (kids), horses (foals) and camels (calves), as further described herein. In an embodiment, the neonatal or young animals are domestic, companion animals, such as, without limitation, dogs and cats of any species. Without wishing to be limiting, “young” animals are generally under one year of age. “Neonatal” animals are generally two weeks of age or less.

The present invention relates to treating diarrhea in neonatal, unweaned and young animals with physiologically and pharmaceutically acceptable formulations and compositions comprising a therapeutically effective amount of an antidiarrheal agent comprising a proanthocyanidin polymer obtained from a Croton spp., preferably Croton lechleri. The proanthocyanidin polymer composition can also be obtained from a Calophyllum spp., in particular Calophyllum inophylum. In an specific embodiment, the pharmaceutically acceptable composition comprises a proanthocyanidin polymer from Croton lechleri. In an specific embodiment, the pharmaceutically acceptable composition comprises a botanical extract derived from Croton lechleri.

In general terms, “treating” an animal according to the present methods refers to achieving or obtaining a desired physiologic and/or pharmacologic effect, whether prophylactic, therapeutic, or both. As used herein “treating” or “treatment” can refer to ameliorating, preventing, inhibiting, reversing, attenuating, alleviating, abrogating, minimizing, suppressing, reducing, decreasing, diminishing, stabilizing, eradicating, curing, or eliminating the deleterious effects of a disease or condition, or the progression or worsening of the disease or condition. For example, successful treatment may involve alleviating one or more symptoms of a disease or condition, although not necessarily all of the symptoms, of the disease or condition, or attenuating the symptoms or progression of the disease or condition. Curing or eliminating the disease or condition from the animal is an optimal outcome of the practice of the methods of the invention.

According to the invention, treatment of an animal in need thereof typically involves the use or administration of an effective amount or a therapeutically effective amount of a proanthocyanidin polymer or a proanthocyanidin polymer composition preferably from a Croton spp., particularly C. lechleri, provided as either an enteric or non-enteric formulation. Effective amount refers to the quantity (amount) of the composition, and the like, that induces a desired response in the animal subject upon administration or delivery to the animal. Optimally, an effective amount produces a therapeutic effect in the absence of, or with little or virtually no, adverse effects or cytotoxicity in the animal. Alternatively, any adverse effects associated with an effective amount are optimally outweighed by the therapeutic benefit achieved.

The treatment methods are directed to ameliorating, preventing, inhibiting, reversing, attenuating, alleviating, abrogating, minimizing, suppressing, reducing, decreasing, diminishing, stabilizing, eradicating, curing, or eliminating diarrhea and/or its associated symptoms caused by a variety of different agents or environmental factors and influences that adversely affect the health, growth and survivability of neonatal and young animals. In an embodiment, the diarrhea is secretory/watery diarrhea. Such diarrhea can be a clinical sign of gastrointestinal (GI) disease in an animal; it can also reflect primary disorders outside of the digestive system, such as disorders affecting the large bowel or the small bowel. The methods described herein are suitable for treating diarrhea resulting from different mechanisms involved in the pathogenesis of the disorders, for example, osmotic diarrhea, secretory diarrhea, episodic diarrhea, or inflammatory and infectious diarrhea. In an embodiment, the neonatal or young animal can suffer from diarrhea associated with inflammation of the lining of the colon, such as colitis, or acute colitis, which can be caused by infection or inflammation of the bowel.

Osmotic diarrhea is associated with absorption of water in the intestines, which depends upon adequate absorption of solutes. If excessive amounts of solutes are retained in the intestinal lumen, water will not be absorbed and diarrhea results. Osmotic diarrhea typically results from ingestion of a poorly absorbed substrate, for example, a carbohydrate or divalent ion or from malabsorption of any type, such as an inability to absorb certain carbohydrates. Secretory diarrhea occurs when the secretion of water into the lumen of the intestine exceed absorption. Under normal conditions, large volumes of water are secreted into the small intestinal lumen, but a large portion of this water is efficiently absorbed before reaching the large intestine.

Secretory diarrhea can result from exposure of an animal to toxins (enterotoxins) from certain types of bacteria, such as cholera toxin of Vibrio cholerae and heat-labile toxin of E. coli. Massive diarrhea is induced from such microorganisms as a consequence of their toxins strongly activating adenylyl cyclase, which causes a prolonged increase in the intracellular concentration of cyclic AMP within crypt enterocytes. This increase. in turn. results in prolonged opening of the chloride channels that contributes to secretion of water from the crypts, thereby allowing uncontrolled secretion of water. These bacterial toxins can also affect the enteric nervous system, resulting in an independent stimulus of water secretion.

Inflammatory and infectious diarrhea can be caused by the disruption of the epithelium of the intestine due to microbial or viral pathogens. Typically, the epithelium of the digestive tube is protected from insult by a number of mechanisms that constitute the gastrointestinal barrier. However, the gastrointestinal barrier can be breached and result in diarrhea. Destruction of the epithelium results not only in leaking of serum and blood into the lumen but also is often associated with significant destruction of adsorptive epithelium. When this occurs, the absorption of water becomes highly inefficient and diarrhea results. The pathogenic culprits frequently associated with infectious diarrhea include bacteria, such as E. coli, Campylobacter and Salmonella; viruses, such as rotaviruses, coronaviruses, parvoviruses and norovirus; and protozoa, such as coccidia species, Cryptosporium and Giardia. In addition, the response of the immune system to inflammatory conditions in the bowel contributes greatly to the development of diarrhea. Activated white blood cells are stimulated to produce and secrete inflammatory mediators and cytokines that stimulate secretion. An secretory component is thus imposed upon and exacerbates an inflammatory diarrhea. Moreover, reactive oxygen species produced by leukocytes can damage or destroy intestinal epithelial cells, which are replaced with immature cells that are generally lacking in the brush border enzymes and transporters necessary for the absorption of nutrients and water. Thus, components of an osmotic (malabsorption) diarrhea provide additional pathology and problems for an afflicted animal.

Thus, in various embodiments, the diarrhea to be treated is caused by infection or invasion of the animals by pathogens, including bacteria, e.g., Escherichia coli, Salmonella spp., Clostridium perfringens, etc.; viruses, e.g., coronaviruses, rotaviruses, bovine virus diarrhea (BVD) virus, infectious bovine rhinotracheitis (IBR) virus, etc.; protozoa, e.g., Cryptosporidium, coccidia, etc.; as well as yeasts and molds. In some cases, diarrhea can be caused by a single infectious microorganism; however, mixed infections, such as caused by, e.g., E. coli plus Cryptosporidium, or Coronavirus plus Salmonella spp., are also not uncommon.

The gram-negative bacterium Escherichia coli is normally found in the intestines of most animals. Although most E. coli are nonpathogenic, some are able to cause intestinal and extraintestinal infections. Large numbers of E. coli are present in the farm environment as a result of fecal contamination. Initial exposure to pathogenic E. coli may occur in contaminated calving pens, but systemic infection usually requires predisposing environmental factors, inadequate transfer of passive immunity or compromised immune system by other infection. The most common type of colibacillosis in young animals is caused by the non-invasive Enterotoxigenic E. coli (ETEC) strains, e.g., K99 STa, which are also the leading cause of diarrhea among travelers and children in the developing world (B. Nagy and P. Z. Fekete, 2005, Int J Med Microbiol., 295:443-454).

Treatment of neonatal and young animals according to the methods of the invention is of particular importance, because such immature animals are most susceptible to infection by numerous pathogens of many types; resistance to infection develops with increasing age of the animal. In addition, younger animals experience more severe clinical illness as a result of infection and resulting diarrhea. For example, young animals (lambs) at one to five days of age experienced more severe infection by enteric cryptospores, causing protracted diarrhea, wasting and death, while young lambs at thirty days of age, which had become infected, did not exhibit severe signs of clinical disease. In general, due to the anatomy of the gastrointestinal tract of adult animals such as horses, conditions affecting the large intestine and cecum typically cause diarrhea. However, young animals, e.g., foals, that are less than about three months of age do not have fully competent large intestines and ceca as do adult animals; therefore, young animals tend to be more prone to diarrhea caused by small intestinal conditions. In general terms, a foal is an equine, particularly a horse, that is one year old or younger in age.

In an embodiment, the diarrhea to be treated results from noninfectious causes, for example, without limitation, inadequate nutrition and/or insufficient attention of the neonate or young animal on the part of the mother, exposure to severe environment, or a combination of these events. In another embodiment, diarrhea results from a combination of the invasion of infectious microorganisms and noninfectious factors. Frequently, noninfectious causes of diarrhea in young animals are considered to be factors that predispose or contribute to an animal's susceptibility to infectious agents and causes of diarrhea. Whether the cause of diarrhea in animals is infectious or noninfectious, the absorption of fluids from the intestine is altered and life-threatening electrolyte imbalances can occur. The affected animals lose fluids, rapidly dehydrate and suffer from electrolyte loss and acidosis. Although infectious agents may cause an initial damage to the animal's intestine, actual death from diarrhea (serious diarrhea) in animals usually is a consequence of dehydration, acidosis and loss of electrolytes, which may be difficult to replenish in adequate amount and time. Accordingly, the methods and formulations of the invention are suitable for treating diarrhea and the symptoms of diarrhea, such as dehydration, weight loss, and electrolyte loss, in an effort to prevent more severe dehydration and animal death.

Because newborn non-human animals, such as calves, are born without a yet functional immune system and without most antibodies that can fight the infectious agents causing sickness and diarrhea in these immature animals, vigilant attention should be paid to the conditions and health of these young animals, particularly during inclement or severe weather conditions and/or difficult births. Neonatal and young animals acquire antibodies from colostrum, which is optimally received by the animals before they are two to four hours old. As young animals grow older, they rapidly lose their ability to absorb colostral antibodies. Thus, for example, colostrum provided to calves that are more than 24 to 36 hours old will likely not be effective, as antibodies are infrequently absorbed following this time in the animal's life.

Due to the unique physiology and susceptibility to diarrheal disease of neonatal animals, prophylactic administration of the C. lechleri proanthocyanidin polymer composition can reduce the incidence of diarrheal disease in neonatal animals, improving health, weight gain and survivability in populations of neonatal animals.

Proanthocyanidins and Tannins Obtained from Plant Extracts

Proanthocyanidins are types of condensed tannins, which are found in a large number of plants and are classified as hydrolyzable or condensed. Tannins and, in particular, proanthocyanidins are contained in many plants used in traditional medicine as treatment or prophylaxis for diarrhea (See, e.g., Yoshida et al., 1993, Phytochemistry, 32:1033; Yoshida et al., 1992, Chem. Pharm. Bull., 40:1997; Tamaka et al., 1992, Chem. Pharm. Bull., 40:2092).

Proanthocyanidins are comprised of at least two or more monomer units that may be of the same or different monomeric structure. The monomer units (generally termed “leucoanthocyanidins”) are generally monomeric flavonoids which include catechins, epicatechins, gallocatechins, galloepicatechins, flavanols, flavonols, flavan-3,4-diols, leucocyanidins and anthocyanidins. The polymer chains are thus based on different structural units, creating a wide variation of polymeric proanthocyanidins and a large number of possible isomers (Hemingway et al., 1982, J. C. S. Perkin, 1:1217). Larger polymers of the flavonoid 3-ol units are predominant in most plants and often have average molecular weights above 2,000 daltons (Da), containing 6 or more units (Newman et al., 1987, Mag. Res. Chem., 25:118).

Proanthocyanidin polymers and proanthocyanidin are found in a wide variety of plants, especially those having a woody habit of growth (e.g., Croton spp. and Calophyllum spp.). A number of different Croton tree species, including Croton sakutaris, Croton gossypifolius, Croton palanostima, Croton lechleri, Croton erythrochilus and Croton draconoides, which are endemic to South America, produce a red viscous latex sap called Sangre de Drago or “Dragon's Blood”. The red viscous latex is known for its medicinal properties. For example, U.S. Pat. No. 5,211,944 describes the isolation of an aqueous soluble proanthocyanidin polymer composition from Croton spp. See also, Ubillas et al., 1994, Phytomedicine, 1:77. The isolation of an aqueous soluble proanthocyanidin polymer composition from Calophyllum inophylum and the use of this composition as an antiviral agent are also described in U.S. Pat. No. 5,211,944.

In an embodiment, a proanthocyanidin polymer from C. lechleri, or a composition thereof, is crofelemer. Crofelemer (CAS 148465-45-6) is an oligomeric proanthocyanidin of varying chain lengths derived from the Dragon's Blood of Croton lechleri, a tree of the family Euphorbiaceae, which is sustainably harvested under fair trade work practices in the Amazon. It has an average molecular weight of approximately 1900 Da to approximately 2700 Da. The monomers comprising crofelemer comprise catechin, epicatechin, gallocatechin, and epigallocatechin. The chain length of crofelemer ranges from about 3 to about 30 units with an average chain length of about 8 units. Crofelemer has the chemical formula: (C₁₅O_{6,7}H₁₂)ₙ and a molecular mass of 860-9100 g/mol. The antisecretory mechanism of action of crofelemer involves the targeting and inhibition of two, distinct intestinal chloride channels, namely, the cystic fibrosis transmembrane regulator conductance (CFTR) channel, which is a cAMP-stimulated Cl⁻ channel, and the calcium-activated chloride channel (CaCC), as reported, for example, by Tradtrantip, L et al., 2010, “Crofelemer, an Antisecretory Antidiarrheal Proanthocyanidin Oligomer Extracted from Croton lechleri, Targets Two Distinct Intestinal Chloride Channels”, Mol. Pharmacol., 77(1):69-78). A general structure of crofelemer is shown below. In the structure, an H at the R position of the structure signifies procyanidin; an OH at the R position of the structure signifies prodelphinidin.

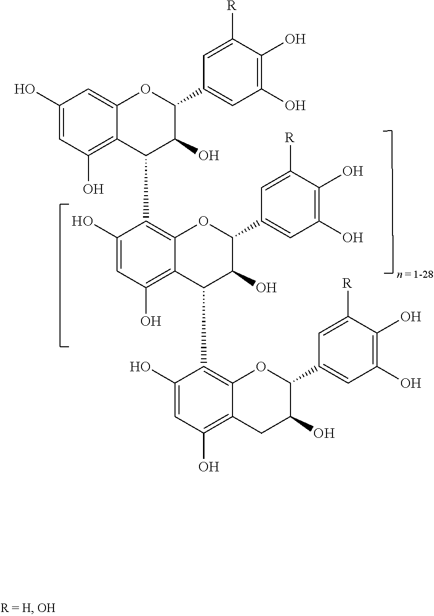

In accordance with an embodiment of the invention, crofelemer, or a pharmaceutically acceptable formulation or composition comprising crofelemer, is employed in the treatment methods as the proanthocyanidin polymer from Croton lechleri.

In an embodiment, SP 303, an oligomeric proanthocyanidin from Croton lechleri, (also known as crofelemer) is the proanthocyanidin polymer from Croton lechleri, or a pharmaceutically acceptable formulation or composition comprising SP 303, which is suitable for use in the treatment methods of the invention. SP-303 (R. Ubillas et al., 1994, Phytomedicine, 1:77-106) is largely composed of purified proanthocyanidin oligomers (−)-galloepicatechin and (+)-gallocatechin, (−)-epicatechin and (+)-catechin and is suitable for use in the enteric and non-enteric formulations and compositions for administration in the treatment methods described herein. The C. lechleri proanthocyanidin may also be isolated according to example 2 of patent application publication US2007/0254050 or in patent application publication US2005/0019389, which are both incorporated by reference herein in their entirety.

In another embodiment, SB 300 is the proanthocyanidin polymer from Croton lechleri, or a pharmaceutically acceptable formulation or composition comprising SB 300, which is suitable for use in the treatment methods of the invention. SB 300, as described, for example, by Fischer, H. et al., (2004, J. Ethnopharmacol., 93(2-3):351-357) provides a natural product extract that is particularly amenable for both enteric and non-enteric formulations and compositions, and is highly functional and cost-effective in the treatment methods described herein.

A pharmaceutically acceptable composition comprising a proanthocyanidin polymer from Croton lechleri and employed in the treatment methods of the invention can be obtained from C. lechleri, e.g., as described in WO 00/47062 to Shaman Pharmaceuticals, Inc., the contents of which are incorporated by reference herein, and formulated as a food or dietary supplement or nutraceutical formulation.

In other embodiments, compositions useful in the methods of the invention comprise a raw latex obtained from a Croton species or a Calophyllum species, or an extract obtained from a Croton species or a Calophyllum species, which are not specifically polymeric proanthocyanidin polymer compositions. Exemplary extracts are described in Persinos et al., 1979, J. Pharma. Sci., 68:124 and Sethi, 1977, Canadian J. Pharm. Sci., 12:7.

In an embodiment, the proanthocyanidin polymer from Croton lechleri is formulated with an enteric coating or matrix in a variety of dosage formats known in the art (See, e.g., WO 00/47062 and U.S. Pat. Nos. 7,441,744 and 7,323,195, the contents of which are incorporated herein, and as briefly described below). In another embodiment, the proanthocyanidin polymer is formulation without an enteric coating or matrix. Both enteric and non-enteric forms of the proanthocyanidin polymer from Croton lechleri, for example, SB 300, are intended for use in the methods of the present invention.

### Preparation of Proanthocyanidin Polymer Compositions and Formulations

The proanthocyanidin polymer composition, effective for treating secretory diarrhea according to the invention, is comprised of monomeric units of leucoanthocyanidins. More particularly, the composition is comprised of proanthocyanidin polymers of 2 to 30 flavonoid units, preferably 2 to 15 flavonoid units, more preferably 2 to 11 flavonoid units and most preferably an average of 7 to 8 flavonoid units with a number average molecular weight of approximately 2500 Da. The proanthocyanidin polymer composition is preferably soluble in an aqueous solution. Preferred for use in the methods according to the invention is a proanthocyanidin polymer from C. lechleri; such a C. lechleri proanthocyanidin polymer may be in the form of a pharmaceutically acceptable composition.

Examples of proanthocyanidin polymeric compositions useful in the present invention are preferably isolated or purified from a Croton spp., namely, Croton lechleri, or Calophyllum spp. by any method known in the art. For example, the proanthocyanidin polymer composition may be isolated from a Croton spp. or Calophylum spp. by the method disclosed in U.S. Pat. No. 5,211,944 or in Ubillas et al. (1994, Phytomedicine, 1:77-106, called SP 303 therein), both of which are incorporated herein by reference. Other isolation methods are described in U.S. Pat. Nos. 7,556,831 and 8,067,041 (Example 2), the contents of which are incorporated by reference herein. PCT application PCT/US00/02687, published as WO 00/47062, the contents of which are incorporated by reference herein, also discloses a method of manufacturing a proanthocyanidin polymeric composition isolated from Croton spp. or Calophyllum spp., and enteric formulations of proanthocyanidin polymer dietary supplements, as well as methods of their preparation. Another illustrative method for isolating proanthocyanidin polymer from C. lechleri (such as crofelemer) is found in U.S. Pat. Nos. 7,341,744 and 7,323,195, the contents of which are expressly incorporated herein. As described above, the SP 303 and SB 300 purified forms of oligomeric proanthocyanidin polymer from Croton lechleri are suitable for use in the treatment methods of the invention.

In an embodiment, the proanthocyanidin polymer composition may be generally isolated by the following process, such as provided in U.S. Pat. No. 7,341,744. Latex collected from Croton lechleri plants is mixed with purified water (preferably one part latex to two parts purified water). Any insoluble material in the latex solution is allowed to settle, e.g., by leaving the mixture at 4° C. overnight (12 hours). The supernatant is pumped away from the residue and is extracted with a short chain alcohol, such as n-butanol. The extraction is preferably performed multiple times, such as three times. After each extraction, the alcohol phase is discarded and the aqueous phase is retained. The aqueous phase is concentrated, for example, using an ultrafiltration device with a 1 kD cut-off membrane. This membrane can be a low protein binding cellulose membrane, or, alternatively, a polypropylene, teflon or nylon membrane can be used. The membrane used should be compatible with acetone. The purpose of the ultrafiltration is to remove the water from the material.

The retentate from the ultrafiltration is then concentrated to dryness, for example using tray-dryers at approximately 37° C. (±2° C.). The dried material is subsequently dissolved in water and is then chromatographed on a cation exchange column (e.g., a CM-Sepharose column) and a size exclusion column (e.g., an LH-20 column). In the preferred two column system, material is run over a CM-Sepharose and then an LH-20 column in a series. Specifically, the dissolved material is loaded onto the cation exchange column and is then washed with purified water. The proanthocyanidin polymer material is eluted from the cation exchange column with an aqueous acetone solution (preferably 30% acetone), thereby loading the proanthocyanidin polymer material onto the sizing column. The sizing column is disconnected from the cation exchange column and the material is then eluted off of the sizing column with an aqueous acetone solution (preferably 45% acetone). The fractions are collected and monitored with a UV detector, e.g., at a wavelength of 460 nm. Fractions containing the proanthocyanidin polymer material are combined and concentrated, for example, by ultrafiltration using, e.g., a 1 kD cut-off membrane (as described above for the ultrafiltration step prior to the chromatography steps). The retentate may then be concentrated to dryness using a suitable drying method, such as, but not limited to, a rotary evaporator, at a temperature of approximately 37° C. (±2° C.). Other suitable drying methodologies include, but are not limited to, tray drying and spray drying. Example 10 of U.S. Pat. No. 7,341,744 provides additional, non-limiting, methodology for preparing a composition comprising proanthocyanidin polymer, which can be used according to the invention. A detailed protocol for isolating an enriched proanthocyanidin polymer extract suitable for use in the methods of the invention is described in WO 00/47062 as noted herein above.

### Methods of Treatment and Applications of Use

The invention is directed to methods of treating diarrhea associated with pathogenic infection and non-pathogenic causes, particularly in neonatal and young animals, comprising administering to an animal in need of such treatment, a pharmaceutically acceptable composition comprising a proanthocyanidin polymer from a Croton species or Calophyllum species in an amount effective to treat the diarrhea. In preferred embodiments, the proanthocyanidin polymer is from a Croton species, namely, Croton lechleri. Treating the diarrhea can involve reducing the severity and duration of the diarrhea in the animal. Treating the diarrhea can also involve increasing the survivability, vigor and weight of the animal, particularly a neonatal or young animal undergoing treatment. In an embodiment, the diarrhea is secretory or watery diarrhea.

The methods of the invention relate to the treatment of non-human animals, notably, but not limited to, the newborns and young of livestock, domestic and farm animals, including grazing animals, which are oftentimes relatively large in size. In one embodiment, the immature animals to which treatment with the proanthocyanidin polymer from Croton lechleri is administered are neonatal (newborn) or infant animals, for example, one to ten hours after birth, one to fifteen hours after birth, twelve to twenty-four hours after birth, twenty-four to thirty-six hours after birth, one to three days after birth, one to four days after birth, one to six days after birth, or one to seven days after birth or up to two weeks after birth. Neonatal animals generally being those under two weeks of age. In an embodiment, the animals are treated between day one and day four after birth. In some embodiments, the neonatal or young animals are treated one to five days of age, less than one week of age, or only a few weeks of age. In an embodiment, treatment occurs during the first weeks of life, for example, one to six weeks of age. In an embodiment, the animals are from two to ten weeks of age, for example, less than one, two, three, four, five, six, seven, eight, nine, or ten weeks of age. The animals undergoing treatment may also be from one to four weeks of age, from one to six weeks of age, or from two to four weeks of age. In some embodiments, the animals are one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days old. In other embodiments, the animals are thirty to forty days old. In other embodiments, the animals are young animals, generally up to one year in age. In many cases, the animals are not weaned (unweaned), i.e., they are still drinking milk. For example, dairy calves are generally weaned at 60 to 80 days while beef cattle may be weaned at 3 to 8 months of age, pigs at 3 weeks of age, dogs at 7 to 8 weeks, and horses at 4 to 6 months of age. Also in many cases, neonatal is synonymous with unweaned. In some cases, the animals are newly weaned or weaned, but still juvenile, young, and non-adult. Such young animals are also highly susceptible to becoming afflicted with diarrhea from various infectious and/or environmental causes.

According to the methods of the invention, the neonatal and young animals can be treated with a proanthocyanidin polymer from C. lechleri, e.g., SB 300, or a botanical extract derived from C. lechleri, for one, two, three, four, five, six, seven, eight, nine, or ten days, etc. The C. lechleri proanthocyanidin polymer can be administered to the animal on consecutive days or intermittently, such as every other day, every two days, every three days, every four days, and the like. In an embodiment, the C. lechleri proanthocyanidin polymer is administered to the animals for three consecutive days. In an embodiment, the C. lechleri proanthocyanidin polymer is administered to neonatal animals between one and four days after birth for three consecutive days. As understood by the skilled practitioner, environmental, e.g., farm, conditions surrounding the neonatal and young animals may dictate the start and course of a treatment regimen such that the administration of the C. lechleri proanthocyanidin polymer occurs earlier in the animal's life and for a longer duration, especially since diarrheal disease typically affects neonatal and young animals in about the first seven days of life, or between about day one or day four of life. In the foregoing embodiments, the animals are bovine or camel calves.

In a particular embodiment, a formulation or composition comprising a botanical extract derived from C. lechleri, SB 300, or SP 303, is provided in the form of a gel or paste formulation that is orally administered to the neonatal or young animal, such as a horse foal, twice daily for three days, preferably, three consecutive days. In a particular embodiment, the twice daily doses are administered to the animal twelve hours apart. The paste formulation is particularly suitable as a product that acts locally in the gut and is minimally absorbed systemically. The paste product specifically addresses the normalization of stool formation and ion and water flow in the intestinal lumen of neonatal and young animals, such as horse foals, and does not alter gastrointestinal motility, i.e., is not constipating. As but one mode of oral delivery, the paste formulation can be placed in the roof of the animal's mouth. In a particular embodiment, the paste formulation comprises beads (nano or microparticles) comprising enterically coated SB 300 or SP 303 and is orally administered to foals. In a particular embodiment, the paste comprising SB 300 enteric beads is orally administered to a foal in need twice daily for three days. In some embodiments, the paste is orally administered for three consecutive days. In a particular embodiment, the paste comprising SB 300 enteric beads is orally administered to a foal at a dose of 2 mg/kg twice daily for three days. The formulation is especially suitable for the normalization of stool formation in a short time period, e.g., less than a week or less than two weeks; for mitigation of weight loss; and reduction in supportive care costs, rehydration therapies, such as oral rehydration, in a young animal afflicted with diarrhea and undergoing treatment.

The types of non-human animals for which the treatment methods are suitable are not particularly limited as to animal type, genus, or species. In general, neonatal or young farm animals, food-source animals, livestock animals, animals bred or kept for various purposes, such as sport (e.g., racing, riding), transport, domestic, companion, industrial uses (e.g. hauling, pulling, plowing), and the like, are particularly amenable to treatment according to the methods of the invention. For example, encompassed by the methods of the invention is the treatment of neonatal or young non-human animals, such as cows (calves), cattle or steer (calves), camels (calves), rams and sheep (lambs), horses (foals), pigs (piglets), goats (kids), bison/buffalo (calves), llamas, donkeys, mules, yaks, etc. Neonatal or young exotic animals, such as zoo animals of various species, are also embraced by the treatments of the invention. In an embodiment, the animals are grazing animals. The treatment of diarrhea in neonates and unweaned animals, for example, calves (bovine, camel, buffalo/bison), lambs, piglets, and foals (equine) is particularly embraced by the described methods.

In accordance with the described methods, the C. lechleri proanthocyanidin polymer composition reduces chloride flux across intestinal epithelial cells and reduces fluid movement into the intestinal lumen, which results in fluid loss and dehydration associated with secretory diarrhea. Therefore, the pharmaceutically acceptable formulations and methods of the invention are useful in prophylactic and therapeutic applications in the treatment of secretory diarrhea, especially in preventing the dehydration and electrolyte loss that accompanies secretory/watery diarrhea.

In a particular embodiment, the methods of the invention treat diarrhea resulting from infection by the Salmonella spp. microorganism with an effective amount of a polymeric proanthocyanidin polymer composition from a Croton species or Calophyllum species, or with a latex, extract or food supplement botanical extract derived therefrom. The treatment of diarrhea caused by Salmonella spp. with a proanthocyanidin polymer composition from Croton lechleri, or with a latex, extract or food supplement botanical extract derived therefrom is an unexpected and surprising aspect of the invention, because Salmonella spp. cause diarrhea by a mechanism of action and by affecting cellular pathways and responses that is distinct and different from the mechanism of action associated with the activity of proanthocyanidin polymer compositions.

More specifically, mechanism of action of polymeric proanthocyanidin polymer compositions, e.g., crofelemer, is through the inhibition of both the cystic fibrosis transmembrane conductance regulator protein (CFTR) chloride ion channel and the calcium-activated chloride ion channels (CaCC). The polymeric proanthocyanidin polymer composition acts by blocking chloride ion channel secretion and the accompanying high volume water loss occurring in diarrhea, thus normalizing the flow of chloride ions and water in the gastrointestinal (GI) tract. However, Salmonella microorganisms trigger diarrhea in infected hosts by producing several virulence factors. One such factor is a protein called SopE, which is injected into intestinal epithelium cells where it triggers a cascade of intracellular signaling events once the bacteria enter the GI tract. (See, e.g., S. Zhang et al., 2003, Infection and Immunity, 71(1):1-12; and A. J. Mueller et al., 2009, Cell Host and Microbe, 6(2):125-136). The binding of the SopE protein to two specific GTPase proteins alters the cell membrane and allows the bacteria to penetrate the cell. In addition, the two GTPase proteins activate Caspase-1 inside the cell, which is a key factor in inflammatory responses. Caspase-1, in turn, causes the production of proinflammatory mediators (cytokines) that attract macrophages which phagocytize the bacteria that has penetrated into the intestinal tissue and cells; however, Salmonella bacteria remaining in the intestinal lumen are not seriously affected. The heightened immune response that exists in the infected animals as a consequence of the infection results in serious inflammation, fluid accumulation and distress for the host animal.

Because Salmonella, which causes a disease pathology and an inflammatory immune response that lead to diarrhea without significantly affecting the CTRF or CaCC, it is considered quite surprising and unexpected that a proanthocyanidin polymer composition which functions by inhibiting these channels is effective in treating diarrhea induced by the Salmonella microorganism. However, the treatment of diarrhea in Salmonella-infected neonatal and young animals, such as, e.g., bovine calves and piglets, with a proanthocyanidin polymer composition (e.g., SB 300) according to present methods demonstrates an unpredicted effectiveness of the composition against diarrhea resulting from a source associated with a different etiology.

In an embodiment, the young animals treated by the methods of the invention are two to four weeks of age. In an embodiment, the animals are two to four week old calves, e.g., without limitation, bovine or camel calves, having diarrhea caused by infection with Salmonella, or crytosporidia or a combination thereof. In an embodiment, the animals are two to four week old calves, e.g., without limitation, bovine or camel calves, having undifferentiated diarrhea of unknown origin. In an embodiment, the animals are horse foals suffering from diarrhea associated with certain adverse environmental conditions and/or infection. In other embodiments, the animals treated by the methods of the invention are approximately 3 to 1000 kg in weight; or approximately 5 to 900 kg in weight, or approximately 10 to 350 kg in weight; or approximately 15 to 150 kg in weight; or approximately 25 to 60 kg in weight, or approximately 30 to 50 kg in weight, or approximately 30 to 40 kg in weight. In an particular embodiment, the young animal being treated for diarrhea is a bovine calf of approximately 20 to 40 kg in weight. In an particular embodiment, the young animal being treated for diarrhea is a camel calf of approximately 30 to 50 kg in weight.

In an embodiment, neonatal and young animals are treated prophylactically with a C. lechleri proanthocyanidin polymer composition, such as SB 300 or SP 303, in enterically protected or non-enterically protected form, to prevent or reduce the risk or severity of the debilitating effects of diarrheal disease and its associated symptoms, e.g., dehydration and weight loss, in neonatal and young animals. According to the treatment method, a C. lechleri proanthocyanidin polymer composition is administered to neonatal and young animals at a suitable time after birth to protect the animals from diarrhea outbreaks typically caused by infections and adverse environmental conditions. Administering a C. lechleri proanthocyanidin polymer composition to neonatal and young animals can also serve to ameliorate or reduce the risk of the animals' suffering from a more serious or severe form of diarrhea relative to animals that are not provided with the C. lechleri proanthocyanidin polymer composition prior to an outbreak of disease or infection. The C. lechleri proanthocyanidin polymer composition can be enteric or non-enteric and can be, for example, SB 300 or SP 303. The dose and regimen of C. lechleri proanthocyanidin polymer composition administration are within the skill of the practitioner to determine and will depend on the environmental conditions and health of the neonatal and young animals to be treated. The animals can be prophylactically treated a with C. lechleri proanthocyanidin polymer composition according to the invention, for example and without limitation, one to seven days, one to six days, one to four days, one to three days, or one or two days after birth. The treatment regimen can involve one, two, three, four, five, six, seven or more days, of C. lechleri proanthocyanidin polymer composition administration to the animals, modified or adjusted as necessary or desired, once or multiple times, e.g., twice, three or four times, per day. The animals can be regularly observed and monitored for health improvements and weight gain. These prophylactic methods of the invention can improve weight gain within the first 15, 20, 25 or 30 days by at least 5%, at least 10%, at least 15%, or even at least 20%.

### Physiologically and Pharmaceutically Acceptable Formulations

The proanthocyanidin polymer from C. lechleri, or a composition thereof, can be provided in any physiologically, pharmaceutically, or therapeutically acceptable form. The pharmaceutically acceptable composition can be formulated for oral administration as, illustratively, but without limitation, powders; crystals; granules; small particles, including microparticles; particles sized on the order of micrometers, e.g., microspheres and microcapsules; particles sized on the order of millimeters, particles sized on the order of nanometers, e.g., nanoparticles; beads; microbeads; pellets; pills; tablets; microtablets; compressed tablets or tablet triturates; molded tablets or tablet triturates; and in capsules, which are either hard or soft and contain the composition as a powder, particle, bead, solution or suspension. The pharmaceutically acceptable composition can also be formulated for oral administration as a solution or suspension in an aqueous liquid, as a liquid incorporated into a gel capsule, as a gel, as a paste or gel paste, or as any other convenient formulation for administration. The composition can be formulated for rectal administration, as a suppository, enema or other convenient form. The proanthocyanidin polymeric composition can also be provided as a controlled release system (See, e.g., Langer, 1990, Science 249: 1527-1533). The composition can be formulated as a dietary supplement or food supplement, e.g., as described in WO 00/47062, for administration to an animal in need thereof according to the present invention.

In a particular embodiment, a formulation or composition comprising a botanical extract derived from C. lechleri, SB 300, or SP 303, is provided in the form of a gel or paste formulation that is orally administered to the adult animal, in need, twice daily for three days, preferably, three consecutive days. In a particular embodiment, the twice daily doses are administered to the animal twelve hours apart. The paste formulation is particularly suitable as a product that acts locally in the gut and is minimally absorbed systemically. The paste product specifically addresses the normalization of stool formation and ion and water flow in the intestinal lumen of the treated animals and does not alter gastrointestinal motility, i.e., is not constipating. As but one mode of oral delivery, the paste formulation can be placed in the roof of the animal's mouth. In a particular embodiment, the paste formulation comprises beads (nano or microparticles) comprising enterically coated SB 300 or SP 303 and is orally administered to the adult animals. In an embodiment, the paste comprising SB 300 enteric beads is orally administered to an animal, such as a horse, twice daily for three days. In some embodiments, the paste is orally administered for three consecutive days. In an embodiment, the paste comprising SB 300 enteric beads is orally administered to an animal, e.g., a horse, in need at a dose of 2 mg/kg twice daily for three days. The formulation is especially suitable for the normalization of stool formation in a short time period, e.g., less than a week or less than two weeks; for mitigation of weight loss; and reduction in supportive care costs, rehydration therapies, such as oral rehydration, in animals undergoing treatment and afflicted with diarrhea.

The pharmaceutical formulation can also include any type of pharmaceutically acceptable excipients, additives, carriers, or vehicles. By way of nonlimiting example, diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose can be added to the proanthocyanidin polymer composition to increase the bulk of the composition. In addition, binders, such as, but not limited to, starch, gelatin, sucrose, glucose, dextrose, molasses, lactose, acacia gum, sodium alginate, extract of Irish moss, panwar gum, ghatti gum, mucilage of isapgol husks, carboxymethylcellulose, methylcellulose, polyvinylpyrrolidone, Veegum and starch arabogalactan, polyethylene glycol, ethylcellulose, and waxes, can be added to the formulation to increase its cohesive qualities. Further, lubricants, such as, but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate and magnesium lauryl sulfate can be added to the formulation. Also, glidants, such as, but not limited to, colloidal silicon dioxide or talc can be added to improve the flow characteristics of a powdered formulation. Disintegrants, such as, but not limited to, starches, clays, celluloses, algins, gums, crosslinked polymers (e.g., croscarmelose, crospovidone, and sodium starch glycolate), Veegum, methylcellulose, agar, bentonite, cellulose and wood products, natural sponge, cation-exchange resins, alginic acid, guar gum, citrus pulp, carboxymethylcellulose, or sodium lauryl sulfate with starch can also be added to facilitate disintegration of the formulation in the intestine.

In some embodiments, the pharmaceutically acceptable formulations contain the proanthocyanidin polymer composition with an enteric coating, in addition to another pharmaceutically acceptable vehicle. In an embodiment, the proanthocyanidin polymer composition can be directly-compressed into a tablet. The tablet can be without excipients and of pharmaceutically acceptable hardness and friability, optionally, with a lubricant, e.g., without limitation, magnesium stearate, and enteric coated. In another embodiment, the pharmaceutically acceptable compositions containing the proanthocyanidin polymer composition alternatively include one or more substances that either neutralize stomach acid and/or enzymes or are active to prevent secretion of stomach acid. These formulations can be prepared by methods known in the art (See, e.g., methods described in Remington's “The Science and Practice of Pharmacy,” 22nd Edition, Editor-in-Chief: Lloyd V. Allen, Jr., Pharmaceutically acceptable Press, Royal Pharmaceutically acceptable Society, London, U K, 2013; and U.S. Pat. No. 7,323,195).

In an embodiment, the proanthocyanidin polymer composition is formulated with a substance that protects the proanthocyanidin polymer and/or the polymer composition from the stomach environment. For such protection, the proanthocyanidin polymer composition can be enteric coated. Enteric coatings are those coatings that remain intact in the stomach, but will dissolve and release the contents of the dosage form once it reaches the small intestine. A large number of enteric coatings are prepared with ingredients that have acidic groups such that, at the very low pH present in the stomach, i.e. pH 1.5 to 2.5, the acidic groups are not ionized and the coating remains in an undissociated, insoluble form. At higher pH levels, such as in the environment of the intestine, the enteric coating is converted to an ionized form, which can be dissolved to release the proanthocyanidin polymer composition. Other enteric coatings remain intact until they are degraded by enzymes in the small intestine, and others break apart after a defined exposure to moisture, such that the coatings remain intact until after passage into the small intestines. A variety of polymers are useful for the preparation of enteric coatings, and the application of an enteric coating to the proanthocyanidin polymer composition can be accomplished by any method known in the art for applying enteric coatings, as may be found, for example, and without limitation, in U.S. Pat. Nos. 7,323,195 and 7,341,744, incorporated herein by reference.

In another embodiment, the pharmaceutically acceptable composition of the proanthocyanidin polymer composition is formulated as enteric coated granules or powder (microspheres with a diameter of 300-500 microns) provided in either hard shell gelatin capsules or suspended in an oral solution for pediatric administration. The enteric coated proanthocyanidin polymer composition powder or granules can also be mixed with food, particularly for administration to neonatal or young animals. Such preparations may be prepared using techniques well known in the art. In addition, the proanthocyanidin polymer composition granules and powder can be prepared using any method known in the art, such as, but not limited to, crystallization, spray-drying or any method of comminution, preferably using a high speed mixer/granulator, as described, for example and without limitation, in U.S. Pat. No. 7,323,195, incorporated herein by reference.

In other embodiments, the proanthocyanidin polymer composition is in the form of an aqueous suspension in admixture with suitable excipients. Non-limiting examples of excipients that are suitable for the manufacture of aqueous suspension include suspending agents, for example, methylcellulose, sodium carboxymethylcellulose, hydroxypropylmethylcellulose, sodium alginate, polyvinyl-pyrrolidone, gum tragacanth and gum acacia; dispersing or wetting agents, which may be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, such as polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, e.g., sucrose, saccharin or aspartame.

Dispersible powders and granules suitable for the preparation of an aqueous suspension by the addition of water provide the proanthocyanidin polymer composition in admixture with a dispersing or wetting agent, suspending agent and one or more preservatives. Suitable dispersing or wetting agents and suspending agents are exemplified by those stated above. Additional excipients, for example, sweetening, flavoring and coloring agents, may also be present.

In an embodiment, the proanthocyanidin polymer composition is a gel or gel formulation. In an embodiment, the proanthocyanidin polymer composition is a paste formulation. In an embodiment, the paste formulation contains a purified botanical extract derived from C. lechleri. In another embodiment, the paste formulation contains enterically coated beads comprising SB 300 or SP 303. In an embodiment, the paste formulation contains enteric protected SB 300 beads. In an embodiment, the gel or paste is contained or preloaded in a delivery device, such as a syringe, e.g., a needle-less syringe, or other type of applicator or delivery system, especially for oral delivery. A gel or paste formulation is particularly suited for administration to Salmonella spp.-infected neonatal and young foals, but also is applicable for other Salmonella spp.-infected adult and neonatal animals, such as those described herein. In an embodiment, the gel or paste is not contained in a delivery device, but is administered to the roof of the mouth of the animal, particularly one that is too incapacitated or ill to eat or drink, thereby eschewing an oral or other mode of administration. In an embodiment, the gel or paste comprises pH-sensitive polymeric particles, such as microparticles or nanoparticles, to allow for pH-dependent uptake of the active compound into cells and/or the pH-dependent release of the active compound in different pH environments in an animal. Processes for generating granules and particles comprising the C. lechleri botanical extract, proanthocyanidin polymer composition, or a compressible form thereof are as known and practiced in the art, and as provided, for example, in U.S. Pat. No. 7,341,744, the contents of which are incorporated by reference herein. In an embodiment, gels are prepared for oral delivery and contain copolymers, such as poloxamers and Pluronics of different types, e.g., Pluronic F.

In another embodiment, the proanthocyanidin polymer composition is in a paste formulation, preferably for oral administration. For example, an oral paste may comprise, without limitation, an oily vehicle or excipient, such as a hydrophobic oily vehicle, a basifying agent, a flavoring agent and a coloring agent. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, and other natural and organic flavorings known to those skilled in the art. Coloring agents can include, for example, iron oxide or titanium dioxide. Alternatively, the oily vehicle can be liquid paraffin or other suitable waxes, including a thickening agent.

Oily suspensions may be formulated by suspending the C. lechleri proanthocyanidin polymer as active ingredient in a vegetable oil, e.g., arachis oil, olive oil, sesame oil or coconut oil, or in mineral oil, such as liquid paraffin. The oily suspensions may contain a thickening agent, e.g., beeswax, hard paraffin or cetyl alcohol. Oral preparations can include sweetening agents as mentioned above and flavoring agents to improve palatability. Pharmaceutically acceptable preservatives, for example, an anti-oxidant such as ascorbic acid, can also be added to such compositions.

The C. lechleri proanthocyanidin polymer pharmaceutical compositions used in the methods of the invention may also be in the form of an oil-in-water emulsions. The oily phase may be a vegetable oil such as olive oil or arachis oil, or a mineral oil such as liquid paraffin or mixtures of these oils. Examples of emulsifying agents include, without limitation, naturally-occurring phosphatides, e.g., soy bean, lecithin, and esters or partial esters derived from fatty acids and hexitol anhydrides, e.g., sorbitan monooleate, and condensation products of partial esters with ethylene oxide, e.g., polyoxyethylene sorbitan monooleate. Sweetening, coloring and flavoring agents can be included in the emulsions.

Syrups and elixirs containing the C. lechleri proanthocyanidin polymer may also can be formulated with sweetening agents, for example, glycerol, propylene glycol, sorbitol or sucrose. Such formulations may also contain a demulcent, a preservative and flavoring and coloring agents. The pharmaceutical compositions may be in the form of a sterile, orally deliverable or administrable aqueous or oleagenous suspension. This suspension may be formulated according to methods known in the art using suitable dispersing or wetting agents and suspending agents, such as those mentioned above. The sterile pharmaceutical preparation may also be a sterile solution or suspension in a non-toxic parenterally-acceptable diluent or solvent, for example, a solution in 1,3-butane diol. Illustrative, acceptable vehicles and solvents that may be used in the preparations include water, Ringer's solution and isotonic sodium chloride solution. Co-solvents, e.g., ethanol, propylene glycol or polyethylene glycols, may also be included. In addition, sterile, fixed oils, e.g., any bland, fixed oil such as synthetic mono- or diglycerides, are conventionally employed as solvents or suspending media and may be used. In addition, fatty acids, such as oleic acid and the like, may be used in injectable preparations.

### Dosage Forms and Administration

In a particular embodiment for treating diarrhea in neonatal animals, e.g., without limitation, bovine and camel calves, foals, kids, lambs, etc., the proanthocyanidin polymer composition is in powder, e.g., reconstitutable powder, form. The composition may be enterically coated or not enterically coated. In an embodiment, the neonates are less than one week in age. In an embodiment, the neonatal animals are bovine calves or camel calves. In an embodiment, the neonatal animals are afflicted with E. coli-induced secretory diarrhea. In an embodiment, the E. coli causative agent is E. coli K99 Sta. In an embodiment, in addition to infection with E. coli, the animal experiences involvement of a viral infection by rotavirus and/or coronavirus, whose mechanism of action involves infection and subsequent destruction of the cells lining the intestinal tract. Such cells are involved in the digestion and absorption of milk in the animal's gut. By treating diarrhea and associated dehydration in neonatal animals and allowing the animals to survive, the methods of the invention also provide the means for the cellular damage in the intestines of the treated neonates and young animals to be repaired.

In an embodiment, the powder form of the proanthocyanidin polymer composition used for treatment is reconstituted or mixed with liquid, such as oral electrolytes, milk or a milk replacer, water, physiological saline, to produce a liquid form or suspension. Milk replacer is generally a source of protein from different origins (for example, milk from a different species, soy, or eggs) and energy (lactose and fat) given to the calf or other animals to replace milk from the mother. In a specific embodiment, the proanthocyanidin polymer composition is mixed at 200 mg to 800 mg per kg of the powder milk replacer prior to reconstitution. In an embodiment, the powder form of the proanthocyanidin polymer composition is provided in the form of individual dosages in packets, e.g., packaged dosage forms, wherein some number of individual packets are provided for use in a treatment regimen. In certain embodiments, the packaged dosage form contain 50 to 500 mg of the proanthocyanidin polymer composition, preferably, 200 to 300 mg of the proanthocyanidin polymer composition. The number of individual doses that can be packaged and provided together is not intended to be limiting, and can include, for example, one to twenty packaged doses; one to ten packaged doses; two, four, six, eight, ten, or more packaged doses, as well as numbers of packaged doses in-between the foregoing, for efficiency of use, handling and for commercial efficacy. Those skilled in the art will appreciate that due to the higher purity of compositions such as SP-303 or crofelemer and SB 300, more by weight of SB 300 than SP-303 will need to be used in formulations to achieve the same amount of the active ingredient of the proanthocyanidin polymer composition. SB 300 generally has about 67% by weight of the proanthocyanidin polymer composition while SP-303 has higher purity, for example 99-100%.

In another embodiment, the powder form of the proanthocyanidin polymer composition is provided in a container, such as a bag, box, bucket, or pail (e.g., 5 lb. to 25 lb. pails), in which the powder can be in an amount of, for example, 100 grams (g) or more, and can optionally include a measuring device, such as a scoop, cup, spoon, trowel, dipper, or ladle. Such containers encompass, for example, an individual daily dose of the proanthocyanidin polymer composition; or an amount suitable for multiple treatments, e.g., a two-day treatment, three-day treatment, four day treatment, etc. An effective amount of the powder can also be mixed with feed for consumption by the young animals, e.g., calves, in need thereof. Dosages may be 200 to 800 mg per day.

In an embodiment, the proanthocyanidin polymer composition is administered or delivered to a neonatal animal afflicted with diarrhea and in need thereof by providing the compound as a bolus. In an embodiment, the proanthocyanidin polymer composition formulated as bolus. i.e., a pill, capsule, or tablet, is orally administered to the neonatal animals afflicted with diarrhea or symptoms thereof, e.g., calves, foals, lambs and kids, directly in the mouth. In an particular embodiment, the treatment regimen comprises administering a dose of 250 mg of the product, e.g., as embraced by one bolus per sick animal for a determined time period, for example, for one, two, or three or more days. The product can be provided to an animal in need thereof in portions of the complete dose, in which the portions are administered one or two or more times per day. Alternatively, the complete dose can be administered to an animal in need thereof one or two or more times per day. In a particular embodiment, the treatment encompasses a dose of 250 mg given two times a day. In another embodiment, the treatment encompasses an oral bolus dose of 250 mg given two times a day for 3 days. In an embodiment, the dose is the Croton lechleri proanthocyanidin polymer composition, SB 300, in enteric form or in non-enteric form, e.g., a reconstituted powder form.

In an embodiment, the proanthocyanidin polymer composition is in a gel or gel formulation. In an embodiment, the gel is contained or preloaded in a delivery device, such as a syringe or other type of injector or delivery system, especially for oral delivery. In an embodiment, the gel comprises pH-sensitive polymeric particles, such as microparticles or nanoparticles, to allow for pH-dependent uptake of the active compound into cells and/or the pH-dependent release of the active compound in different pH environments in an animal. A gel formulation is particularly suited for administration to neonatal and young foals, but also is applicable for other neonatal animals, such as those described herein. In an embodiment, the gel is not contained in a delivery device, but is administered to the roof of the mouth of the animal, particularly one that is too incapacitated or ill to eat or drink, thereby eschewing an oral or other mode of administration. In an embodiment, gels are prepared for oral delivery and contain copolymers, such as poloxamers and Pluronics of different types, e.g., Pluronic F. Processes for generating granules and particles comprising the proanthocyanidin polymer composition or a compressible form thereof are as known and practiced in the art, and as provided, for example, in U.S. Pat. No. 7,341,744, the contents of which are incorporated by reference herein.

In another embodiment, the proanthocyanidin polymer composition is in a paste formulation, preferably for oral administration. For example, an oral paste may comprise, without limitation, an oily vehicle or excipient, such as a hydrophobic oily vehicle, a basifying agent, a flavoring agent and a coloring agent. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, and other natural and organic flavorings known to those skilled in the art. Coloring agents can include, for example, iron oxide or titanium dioxide. Alternatively, the oily vehicle can be liquid paraffin or other suitable waxes, including a thickening agent. In an embodiment, the paste formulation contains beads with enterically coated SB 300 or SP 303, which is administered to an animal, such as a horse foal, at a dose of 2 mg/kg. More particularly, the paste formulation containing enterically coated SB 300 beads is administered to the foal at a dose of 2 mg/kg, twice a day for three days. In an embodiment, the paste containing enteric protected SB 300 beads is administered twice a day at twelve hour intervals.

The routes of administration of the C. lechleri proanthocyanidin polymer product to afflicted animals are not intended to be limiting. Illustratively, administration can be via any suitable, convenient or preferred route of administration including oral, buccal, dental, periodontal, via food source (animal feed), nutrition source, or libation source, otic, inhalation, endocervical, intramuscular, subcutaneous, intradermal, intracranial, intralymphatic, intraocular, intraperitoneal, intrapleural, intrathecal, intratracheal, intrauterine, intravascular, intravenous, intravesical, intranasal, ophthalmic, biliary perfusion, cardiac perfusion, spinal, sublingual, topical, transdermal, intravaginal, rectal, ureteral, or urethral. In certain embodiments, oral, buccal, and food and/or drink supplement are particularly suitable routes. In an embodiment, the product is an aqueous formulation and is provided to the animal as a drench or directly from a ready-to-use (RTU) bottle directed to the esophageal cavity so as to more effectively reach the animal's intestine/gut for optimal activity. In a related embodiment, administration can also be by inclusion in the regular or special diet of the animal, such as in a functional food for the animals or companion animals.

Dosage forms can include, without limitation, oral, injectable, transdermal, aerosol including metered aerosol, chewable products or pellets, capsules, capsule containing coated particles, nanoparticles, or pellets, capsule containing delayed release particles, capsule containing extended release particles, concentrates, creams and augmented creams, suppository creams, discs, dressings, elixirs, emulsions, enemas, extended release films or fibers, gases, gels, metered gels, granules, delayed release granules, effervescent granules, implants, inhalants, injectable lipid complexes, injectable liposomes, inserts or devices, extended release inserts, intrauterine devices, jellys, liquids, extended release liquids, lotions, augmented lotions, oils, ointments, augmented ointments, pastes, pastilles, pellets, powders, reconstituted powders, extended release powders, metered powders, solutions, drops, concentrated solutions, gel forming solutions/drops, sponges, sprays, metered sprays, suppositories, suspensions, suspensions/drops, extended release suspensions, syrups, tablets/pills, chewable tablets/pills, tablets/pills containing coated particles, delayed release tablets/pills, dispersible tablets/pills, effervescent tablets/pills, extended release tablets/pills, orally disintegrating tablets/pills, tapes, or troches/lozenges. The dosages can be provided as formulations, compositions, pharmaceutically acceptable formulations and compositions, physiologically acceptable formulations and compositions, including pharmaceutically and physiologically acceptable carrier, excipients, diluents, or vehicles as known and used in the art.

For oral administration, the C. lechleri proanthocyanidin polymer product, or a composition thereof, is preferably encapsulated and formulated with suitable carriers, and the like, in solid dosage forms. Nonlimiting examples of suitable carriers, excipients, diluents and vehicles include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, edible oils, and the like. The formulations can also include lubricating agents, wetting agents, emulsifying and suspending agents, preserving agents, sweetening agents or flavoring agents. The compositions can be formulated to provide rapid, sustained, extended, or delayed release of the active ingredient after administration to the animal by employing protocols and methods well known in the art. The formulations can also include compounds or substances that reduce proteolytic degradation and promote absorption such as, for example, surface active agents.

As will be appreciated by those having skill in the art, the specific dose can be calculated according to the approximate body weight, body mass, or body surface area of the animal, or the volume of body space or mass to be occupied. The dose also depends on the particular route of administration selected by the practitioner. Further refinement of the calculations necessary to determine an appropriate dosage for treatment is routinely made by those of ordinary skill in the art, for example, using appropriate assays and analytical procedures, such as has been described for certain compounds (e.g., Howitz et al., Nature, 425:191-196, 2003). Exact dosages can be determined based on standard dose-response studies. Therapeutically effective doses for treatment of afflicted animals can be determined, by titrating the amount of the active product given to the animal to arrive at the desired therapeutic effect, while minimizing side effects.

For use in treating diarrhea, such as secretory or watery diarrhea, and its symptoms in neonatal and young animals in accordance with the methods of the invention, a therapeutically acceptable form of the C. lechleri proanthocyanidin polymer composition, including a C. lechleri botanical extract, is administered, particularly orally administered, in an amount ranging from 0.1 to 100 mg/kg per day, once, twice or more daily. In other embodiments, the amount can range from about 0.1 to about 10 mg/kg/day, once, twice or more daily; or from about 0.1 to about 25 mg/kg/day, once, twice or more daily; or from about 0.1 to about 30 mg/kg/day, once, twice or more daily; or from about 0.1 to about 40 mg/kg/day, once, twice or more daily. In other embodiments, the dose can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, etc., as well as incremental dose amounts in between. In still other embodiments, the amount can range from about 1 to about 10 mg/kg/day once, twice or more daily; or from about 1 to about 5 mg/kg/day, from about 1 to about 8 mg/kg/day, from about 1 to about 10 mg/kg/day, or from about 2 to about 4 mg/kg/day once, twice or more daily. In an embodiment, the amount of the C. lechleri proanthocyanidin polymer composition for administration is 2 mg/kg two times a day. In an embodiment, the 2 mg/kg dose is administered twice a day for three days. In a more particular embodiment, SB 300 enteric beads are formulated in a paste which is administered to a neonatal or young animal, e.g. a horse foal, at a dose of 2 mg/kg two times a day for three days. In other embodiments, the foregoing amounts of the C. lechleri proanthocyanidin polymer composition are administered, for example, twice daily, three times daily, four times daily, or more than four times daily, rather than once per day. Higher doses, e.g., 50 mg/kg or 100 mg/kg per day or twice or more daily, may be required, as necessary, to treat diarrhea and accompanying dehydration in the neonatal and young animals.

In other embodiments, for the treatment methods, a suitable dose for the C. lechleri proanthocyanidin polymer product, or the C. lechleri proanthocyanidin polymer composition, such as SP 303 or SB 300, may range from about 1 mg to about 1000 mg, either daily or multiple times per day. In an embodiment, a suitable dose may range from about 10 mg to about 500 mg, either daily or multiple times per day. In an embodiment, a suitable dose may range from about 50 mg to about 350 mg, either daily or multiple times per day. In an embodiment, a suitable dose may range from about 30 mg to about 400 mg, either daily or multiple times per day. In an embodiment, a suitable dose may range from about 100 mg to about 250 mg, either daily or multiple times per day. In an embodiment, a suitable dose may range from about 50 mg to about 300 mg, either daily or multiple times per day. It will be understood that the ranges include the lower and higher amounts specified, as well as amounts in between. The doses administered multiple times per day can be given for consecutive days, e.g., two days, three days, four days, five days, six, days, seven days, or more, in some embodiments. A dose administered multiple times per day may embrace two, three, four, five, six, or more times per day. Other dosing schedules, such as every other day, or every third day, every fourth day, etc. are embraced by the invention. In addition, one having skill in the art will appreciate that doses and amounts administered to the animal can vary, given the wide range of weights of the animals undergoing treatment, as well as the animal species and type of digestive system, e.g., ruminant or non-ruminant. In an embodiment the C. lechleri proanthocyanidin polymer is SB 300. In an embodiment the C. lechleri proanthocyanidin polymer is enterically coated SB 300. In an embodiment the C. lechleri proanthocyanidin polymer is non-enterically coated SB 300.

In some embodiments, daily doses, including multiple daily doses, e.g., twice or three times a day, of the C. lechleri proanthocyanidin polymer product may be 2 mg, 5 mg, 10 mg, 15 mg, 20 mg, 25 mg, 50 mg., 100 mg, 150 mg, 175 mg, 200 mg, 225 mg, 250 mg, 275 mg, 300 mg, 325 mg, 350 mg, 375 mg, 400 mg, 500 mg (or there between) per animal. Administration schedules may also be altered to achieve a therapeutically effective concentration of the C. lechleri proanthocyanidin polymer to treat the diarrhea and its symptoms as described herein. By way of specific, yet nonlimiting example, a suitable dosage amount for use in the methods according to the invention is 250 mg administered once or twice daily. In some embodiments, the compound may be administered once per day, twice per day, thrice per day, 4 times per day, 5 times per day, 7 times per day or 10 times per day. Often the dosage is divided into equal parts administered throughout the day, however in some embodiments related to treating more severe or entrenched symptoms, it may be useful to tailor the dosage administration schedule so that most of the daily treatment is administered at a predetermined time of the day, e.g., the beginning half of the day. In some embodiments, about 50% 60%, 70% or 80% of the dosage is administered in the first half of the day. In other embodiments, it may be more appropriate to administer most of the dosage in the latter half of the day so that about 50%, 60%, 70% or 80% of the dosage is administered in the latter half of the day.

It will be understood that the dose amount actually administered can be determined by the practitioner, in the light of the relevant circumstances, including the severity of the disease, condition, or symptoms thereof being treated, the form of the product to be administered, the age, weight, and response of the individual animal receiving treatment, as well as the chosen route of administration.

The methods of the invention further embrace the administration of pharmaceutically acceptable formulations of the proanthocyanidin polymer composition either alone or in combination with other supplements or agents for treatment or amelioration of the symptoms of secretory diarrhea, such as rehydration agents, electrolytes (e.g., sodium, potassium, magnesium, chloride and formulations thereof), antibiotics, gut-lining protectants, such as kaolin, pectin, or bismuth liquid, and fluid adsorbents, such as attapulgite. Other agents may include anti-motility agents, although because many of the microorganisms and pathogens that are associated with diarrhea induction in neonatal and young animals concomitantly decrease gut motility, the use of anti-motility drugs may be contraindicated. Natural biological products, e.g., Lactobacillus, Bifidobacterium, or Streptococcus faecium, other bacteria and yeast microorganisms, or probiotics, may also be employed as additives to restore the natural balance of intestinal flora in the affected neonatal animals. Such natural biological products, e.g., probiotics as known in the art, may be administered in conjunction with the C. lechleri proanthocyanidin polymer or composition thereof, for example, prior to, at the same time as, or after the administration of the proanthocyanidin polymer or composition to a non-human animal. In addition, a reconstituted C. lechleri proanthocyanidin polymer or composition thereof may include probiotics in accordance with the present invention.

### Exemplary Specific Embodiments Encompassed by the Invention

The present invention is further directed to uses and methods encompassed by the following embodiments.

In an embodiment, the invention provides the use of a composition comprising an aqueous soluble proanthocyanidin polymer from Croton lechleri in treating diarrhea associated with enteropathogenic infection in a neonatal or young non-human animal, wherein the composition is formulated as a pharmaceutically acceptable bolus or reconstituted powder and is orally administered to the neonatal or young non-human animal in an amount of at least 40 mg to 300 mg per day for two or more consecutive days. In another embodiment, the invention provides a method of treating a neonatal or young non-human animal having diarrhea associated with enteropathogenic infection, the method comprising orally administering to the neonatal or young non-human animal in need thereof a pharmaceutical composition comprising an aqueous soluble proanthocyanidin polymer from Croton lechleri, wherein the composition is formulated as a bolus or as a reconstituted powder and administered to the neonatal or young non-human animal in an amount of at least 40 mg to 300 mg per day for two or more consecutive days. In an embodiment, the enteropathogenic infection is with one or more of E. coli, rotavirus, or coronavirus, and, optionally, wherein the enteropathogenic infection is additionally with Salmonella spp. and/or Cryptosporidia.

In an embodiment, the invention provides the use of a composition comprising an aqueous soluble proanthocyanidin polymer from Croton lechleri for improving weight gain or reducing mortality in a neonatal or young non-human animal, wherein said composition is formulated as a pharmaceutically acceptable bolus or reconstituted powder and is administered to the neonatal or young non-human animal in an amount effective to improve weight gain or reduce mortality for at least one day between the first and fourth days after birth of said neonatal or young non-human animal. In another embodiment, the invention provides a method of improving weight gain or reducing mortality in neonatal non-human animal, said method comprising administering to said neonatal non-human animal a pharmaceutical composition comprising an aqueous soluble proanthocyanidin polymer from Croton lechleri, wherein the composition is formulated as a bolus or as a reconstituted powder and administered to the neonatal or young non-human animal in an amount effective to improve weight gain or reduce mortality for at least one day between the first and fourth days after birth of said neonatal non-human animal.

In embodiments related to the above uses and methods, the neonatal or young non-human animal is selected from the group consisting of a bovine calf, a camel calf, a buffalo calf, a bison calf, a lamb, a kid, a foal and a piglet. In a particular embodiment, the neonatal or young non-human animal is a bovine calf or a camel calf. In another embodiment, the neonatal or young non-human animal is characterized as being one or more of: (i) unweaned; (ii) less than two weeks of age; (iii) two to four weeks of age; (iv) approximately 30 to 50 kg in weight; (v) approximately 30 to 40 kg in weight; or (vi) approximately 40 to 50 kg in weight. In other embodiments, the C. lechleri proanthocyanidin polymer composition (i) is administered in a as a powder reconstituted with oral electrolytes, milk or a milk substitute, physiological saline, or water; (ii) is administered as a bolus; or (iii) is administered in animal feed. In an embodiment, the C. lechleri proanthocyanidin polymer composition is administered for at least three consecutive days. In another embodiment, the proanthocyanidin polymer composition is administered twice daily for at least three consecutive days. In an embodiment, the composition is administered to the neonatal or young non-human animal in an amount of at least 30 mg to 350 mg. In an embodiment, the proanthocyanidin polymer composition is administered to the neonatal or young non-human animal in an amount of 250 mg. In a particular embodiment, the neonatal or young non-human animal is a lamb or a kid, and the composition is administered in an amount of 40 mg or 50 mg.

In an embodiment, the invention further provides the use of a composition comprising an aqueous soluble proanthocyanidin polymer from Croton lechleri in treating diarrhea in a neonatal or young equine animal, wherein the composition is provided as a pharmaceutically acceptable bolus, reconstituted powder, or a gel and is administered to the animal in an amount of at least 100 mg for two or more consecutive days. In another embodiment, the invention provides a method of treating a neonatal or unweaned equine animal for diarrhea associated with enteropathogenic infection, the method comprising orally administering to the animal a pharmaceutical composition comprising an aqueous soluble proanthocyanidin polymer from Croton lechleri, wherein the composition is provided in a form selected from a bolus, a reconstituted powder, or a gel, and is administered to the animal in an amount of at least 100 mg for two or more consecutive days. In an embodiment, the animal is infected with bacteria, viruses and protozoa, which infection induced the diarrhea. In an embodiment, the C. lechleri proanthocyanidin polymer composition is administered to the animal in an amount of at least 250 mg. In an embodiment, the gel comprises polymeric microparticles or nanoparticles containing the composition. In an embodiment, the polymeric microparticles or nanoparticles are pH-sensitive. In another embodiment, the neonatal or young animal is less than two weeks of age and/or is approximately 30 to 50 kg in weight.

In an embodiment, the invention further provides the use of a composition comprising an aqueous soluble proanthocyanidin polymer from Croton lechleri in treating or preventing diarrhea in a neonatal or young equine foal, wherein the composition is formulated as a pharmaceutically acceptable paste and is orally administered to the foal in an amount of 2 mg/kg per day two times per day for two or more consecutive days. In an embodiment, the invention is directed to a method of treating or preventing diarrhea in a neonatal or young equine foal, the method comprising orally administering to the foal in need thereof a pharmaceutical composition comprising an aqueous soluble proanthocyanidin polymer from Croton lechleri, wherein the composition is formulated as a paste and is orally administered to the foal in an amount of 2 mg/kg per day two times per day for two or more consecutive days. In an embodiment, the neonatal or young equine foal is unweaned. In an embodiment, the C. lechleri proanthocyanidin polymer composition is formulated as a paste and is orally administered to the foal in an amount of 2 mg/kg per day two times per day for three consecutive days. In an embodiment, the paste is administered two times a day, twelve hours apart, for three consecutive days. In an embodiment, the paste is contained in a delivery device, which is optionally a syringe. In an embodiment, oral administration comprises applying the paste to the roof of the animal's, e.g., foal's, mouth.

In an embodiment related to the above uses and methods, the composition comprising the C. lechleri proanthocyanidin polymer is administered to the animal as an enteric coated pharmaceutical composition or as a non-enteric coated pharmaceutical composition. In other embodiments, the composition comprising the C. lechleri proanthocyanidin polymer is SB 300, SP 303, or crofelemer.

The following examples describe the invention in its various aspects and are not intended to be limiting.

### EXAMPLES

### Example 1

### Control of Diarrhea in Neonatal Camel Calves Treated with a Composition Containing a Proanthocyanidin Polymer or Oligomer Extract from Croton lechleri

Neonatal diarrhea remains one of the most common causes of death in young camels. Enterotoxigenic E. coli (ETEC) and rotavirus appear to be the most significant infectious causes of diarrhea during the first week of a newborn camel's life. Salmonella is also a problem in older calves. The pathophysiology includes induction by toxins of the secretion of water in the small intestine with secretory diarrhea as a result. Regardless of the pathogens involved in the disease process, treatment is aimed at preventing and correcting the resulting fluid and electrolyte deficits. Calves can lose 5 to 10% of their body weight in water in one day of scouring. It is therefore crucial to limit water loss.

The goal of this study is to confirm the efficacy of a Croton lechleri proanthocyanidin polymer extract composition (NSF) from Napo Pharmaceuticals Inc. in controlling secretory diarrhea in young camel calves (<6 weeks old) in a preliminary study.

Animals:

Thirty (30) young calves aged between (1 and 6 weeks) from both genders with clinical signs of diarrhea are enrolled in the study. They are randomly allocated to a treatment group or a control group. Twenty (20) are treated with NSF and ten (10) serve as controls.

Exclusion/Inclusion Criteria:

Only calves less than 6 weeks of age with clinical signs of diarrhea are included in the study. Calves with signs of respiratory disease or arthritis are excluded from the study as well as animals showing signs of diarrhea for more than four (4) days.

Treatment Other than Test Articles:

Electrolytes and fluids either oral or IV are administered at the discretion of the attending veterinarian or caretaker. The quantity and frequency are recorded on the data sheet. Antibiotics and NSAIDS may also be used at the discretion of the veterinarian, if they are the “standard of care” treatment The quantity and frequency of their use are recorded on the data sheet. Intestinal protectants and absorbents such as kaolin, activated attapulgite, activated charcoal should not be used, or the animal should be withdrawn from the study. Gastrointestinal modality modifiers (such as loperamide, hyoscine, atropine, dipryrone) are not used in this study.

Test Articles:

Croton lechleri extract (NSF) in a small bolus (pill) is administered to the calves directly in the mouth. Sick animals are treated twice a day for 3 days with a dose of 250 mg of product (one pill) per calf, together with the “standard of care treatment” (2 liters of oral electrolyte twice a day or 4 liters of intravenous fluid per day). Treatment group allocation in chronological order includes: the 2 first animals will be treated (from Group 1), the third is a control (from Group 2), 2 treated, one control, and so on. The controls receive the standard of care treatment without the NSF pill. The “standard of care” treatment is the same for both groups.

Clinical Examination:

The animals in the study are examined twice a day for 3 days and scored using the parameters detailed below in Table 1.

TABLE 1

| Parameters | Score |
|---|---|
| 1. Posture/Ability to Stand: | |
| Animal standing up by itself, alert and active | 1 |
| Animal standing after encouragement | 2 |
| Animal standing steadily after lifting | 3 |
| Animal standing unsteadily | 4 |
| Animal unable to stand, in sternal or lateral recumbancy | 5 |
| 2. Degree of Enophthalmos: | |
| Normal | 1 |
| Slightly sunken (visible gap between globe and carencula | 2 |
| lacrimalis but less than 2 mm) | |
| Severely sunken | 3 |
| 3. Suckling Reflex | |
| Strong | 1 |
| Weak | 2 |
| Absent | 3 |
| 4. Hydration by Skin Tenting: | |
| Pinch a fold of skin on the neck and count the seconds it takes | |
| to flatten: | |
| Two seconds = normal | 1 |
| 2 to 6 seconds = 8% dehydration | 2 |
| More than 6 seconds = severe dehydration 10% | 3 |
| 5. Diarrhea: | |
| Normal feces, consistence of pudding sample retains original | 1 |
| shape if placed in a container | |
| Semi-solid, less firm such as yogurt. Sample spread across the | 2 |
| bottom of the container but it is not liquid | |
| Sample is liquid with the consistency of maple syrup | 3 |
| Consistency of apple juice but some fecal matter still seen. | 4 |
| Consistency of water, no fecal matter, some mucus or blood | 5 |
| could be seen | |
| 6. Body Temperature: | |
| Data will be recorded on the data sheet provided. | |

### Example 2

### Evaluation of the Effect of Oral Administration of a Croton lechleri Proanthocyanidin Polymer Composition on the Fecal Scores of Salmonella typhimurium-Infected Neonatal Bovine Calves Afflicted with Diarrhea

Diarrhea remains an important cause of morbidity and mortality in neonatal calves (P. Constable, 2004, J Vet Intern Med., 18:8-17). The economic losses associated with this disease are due not only to the resulting mortality, but also to the retarded growth of the animals, the cost of both the veterinary care and the drugs used to treat the infection, and the increased labor involved (D. C. de Graaf et al., 1999a and 1999b, Int J Parasitol., 29:1269-1287 and 1289-1306). Several enteropathogens are associated with diarrhea in neonatal calves, the most prevalent being Escherichia coli, Clostridium perfringens, Salmonella spp., Cryptosporidium spp., and rotavirus and coronavirus, with their relative importance varying by geographic region (D. R. Snodgrass et al., 1986, Veterinary Record, 119:31-34; E. E. Younis et al., 2009, Res Vet Sci., 87:373-379).

A small completely randomized study was conducted to evaluate the effect of oral administration of 250 mg of a Croton lechleri proanthocyanidin polymer composition, i.e., oral SB 300, on fecal consistency of bovine calves infected with Salmonella typhimurium and receiving treatment twice daily for 3 consecutive days. Fecal consistency scores were determined throughout the treatment period. A total of 82 calves were randomly allocated into one of two treatment groups; 39 calves were allocated into the control groups and 43 calves were allocated into the treatment groups. All calves were clinically affected with diarrhea induced by Salmonella infection and received palliative therapy according with the farm standard operating procedures. In addition, calves allocated the treatment group received the same palliative care and were treated orally with the Croton lechleri proanthocyanidin polymer SB 300 composition, as above.

Calves were scored for fecal consistency using a three level score system; 0=solid/normal well-formed feces, 1=pasty feces, and 2=watery diarrhea. Each calf received a total of 6 fecal scorings (twice daily, morning and afternoon) for three days following the diarrhea diagnosis. Data were analyzed using repeated measures ANOVA.

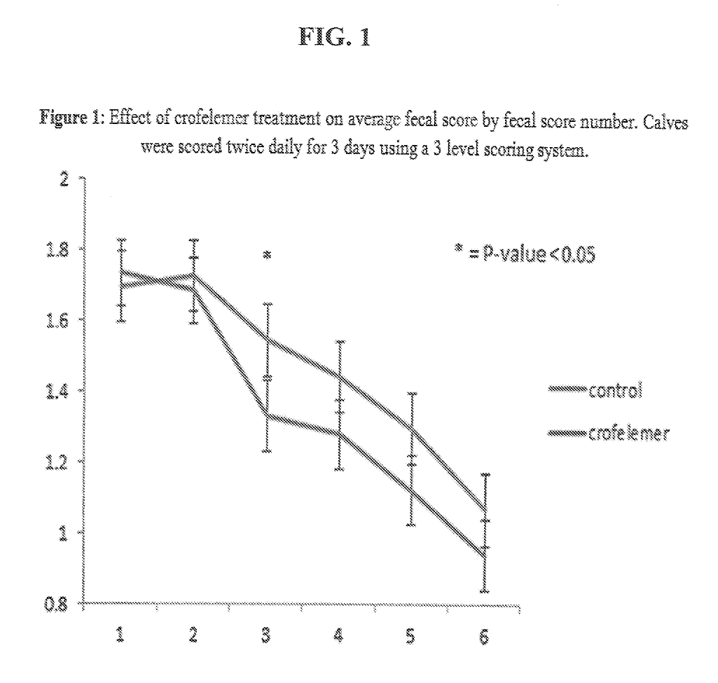

Treatment with the Croton lechleri proanthocyanidin polymer extract composition (SB 300) had a strong tendency to improve (i.e., decrease) fecal scores (P value=0.05). Overall, the average fecal score for control calves was 1.46 and for the treatment calves it was 1.34 (P value=0.05). Fecal consistency scores were similar between treatment group at the beginning of the study. Calves treated with the Croton spp. proanthocyanidin polymer extract composition demonstrated faster improvement on diarrhea scores, starting on the second day of treatment (See, FIG. 1). The study results demonstrate that calves having malabsorption-type diarrhea resulting from infection with Salmonella bacteria showed improvement in fecal scores, indicating the successful treatment of diarrhea, when treated with the SB 300 composition.

### Example 3

### Treatment of E. coli Challenged Calves with a Croton lechleri Proanthocyanidin Polymer Extract Composition SB 300

This Example describes a bovine calf clinical study that was conducted in the isolation unit at Cornell University, Ithaca, N.Y., in which the calves were treated with either enteric or non-enteric formulations of crofelemer, the Croton lechleri proanthocyanidin polymer extract composition. All calves were male Holsteins from the same dairy farm in upstate New York. The calves' weights at birth ranged from 57 pounds to 106 pounds.

In this study, calves were clean caught and within two hours were transported to an isolation facility for research animals (Cornell Animal Research Facility, Ithaca, N.Y.). Calves were individually housed in 16 square meter rooms with controlled temperature and humidity. For the clinical trial, the calves were challenged using an enterotoxigenic E. coli serotype 09:K35:K99 (ATCC #31616). After standard bacterial activation, E. coli serotype 09:K35:K99 were grown in Trypticase soy broth (BBL Microbiology Systems) for 8 hours and then on Minca-IsoVitaleX (BBL) agar for 18 hours at 37° C. The bacteria were suspended in phosphate-buffered saline with 10% dimethyl sulfoxide and stored in 10 mL aliquots at −70° C. The mean inoculum titer was 4×10¹⁰ colony-forming units (CFU) per 10 ml (CFU/10 mL).

All calves were challenged at the research facility within 5 hours of life, e.g., 1 to 5 hours after birth. A mixture of freshly-prepared 1 liter (L) of antibiotic-free colostrum replacer plus 10 mL of thawed E. coli inoculum, described above, was administered to the calves via esophageal feeder. Calves were fed non-medicated milk replacer (22-20) on a 10% body weight daily basis, twice a day, e.g., at 0600 h and 1800 h. All calves were kept in the study until 25 days of life with ad libitum access to water. In addition, calf starter (Calf starter 18% CP, DuMOR®, Tractor Supply Co.) was also available ad libitum starting on the seventh day of life.

In this study, 60 calves were enrolled in three groups. Group 1: Twenty (20) calves received 250 mg of the Croton lechleri proanthocyanidin polymer extract composition SB 300 as an enteric coated tablet twice a day for three days. Group 2: Twenty one (21) calves received 250 mg SB 300 as a non-enteric powder reconstituted (dissolved) in milk or oral electrolytes twice a day for three days. Group 3: Nineteen (19) calves received a placebo (enteric coated tablets containing sugar and iron oxide), either as a tablet or a reconstituted powder twice a day for three days, as control.

Treatments were administered twice daily before each meal (a total of six treatments per calf), with the first treatment administered before the first meal (approximately 12 hours after bacterial challenge). Calves were weighed at birth and again at 10, 15 and 25 days of life. Fecal scores (plus 20 grams of fecal sample were collected), skin turgor and eyes recession (indicative of dehydration) were evaluated twice daily for each calf, from birth until day 10, at day 15, and at day 25 of life.

In general, diarrhea was treated according to the dehydration level and attitude of the calf. Calves having diarrhea and slight dehydration, but having normal appetite, were offered oral electrolytes (Re-sorb, Pfizer); calves affected with diarrhea, dehydration, and poor appetite, but still ambulatory, were fed 2 liters of oral electrolyte mix; and calves unable to stand and severely dehydrated were treated with 4 liters of intravenous fluid (Plasma-lyte 148 and 5% dextrose injection; Baxter Corporation).

Calves were closely monitored for dehydration, appetite, attitude, fecal consistency and any adverse health disorder (Table 2). A first blood sample was collected from all study calves within 12 hours post challenge. For every calf, fecal samples were collected twice daily in order to analyze dry mater of the feces, and a daily blood sample collection was performed to monitor precisely the hydration status (total protein, packed cell volume (PCV), chemistry). Daily starter intake and milk intake were recorded for the entire study period. Immunoglobulin-G levels were measured for blood collected 48 hour after birth using an ELISA kit (Bethyl Laboratories, (Montgomery, Tex.). For all daily blood samples; serum total protein, PCV and the acid-base serum status (full blood chemistry) were conducted at the Clinical Pathology laboratory (Cornell University, Ithaca, N.Y.). Calf health-related events used as criteria for clinical diagnosis and assessment of animal health are presented in Table 2 below.

Fecal scores (Table 2) were evaluated as follows: the scores were based on diarrhea severity and a 5 point scale to assess visually calf diarrhea on milk-fed calves. Fecal scores were categorized as follows: 0=formed feces with normal color; 1=pasty (semiformed) feces with normal color; 2=liquid (watery) feces with normal color; 3=watery feces with normal color (and/or mucous); and 4=watery feces with abnormal color (and/or blood in feces).

TABLE 2

| Health condition | Clinical signs | Score |
|---|---|---|
| Fecal Consistency | Formed | 0 |
| | Semiformed | 1 |
| | Watery | 2 |
| | Watery with mucous | 3 |
| | Blood in feces | 4 |
| Hydration | Normal appearance | 0 |
| | Sunken eyes | 1 |
| | Skin tented 5 to 10 s | 2 |
| | Skin tented ≧10 s | 3 |
| Attitude | Alert | 0 |
| | Depressed | 1 |
| | Non responsive | 2 |
| Appetite | Normal | +++ |
| | Consuming <½ bottle | ++ |
| | Consuming <¾ bottle | + |
| | (orogastric tube) | |

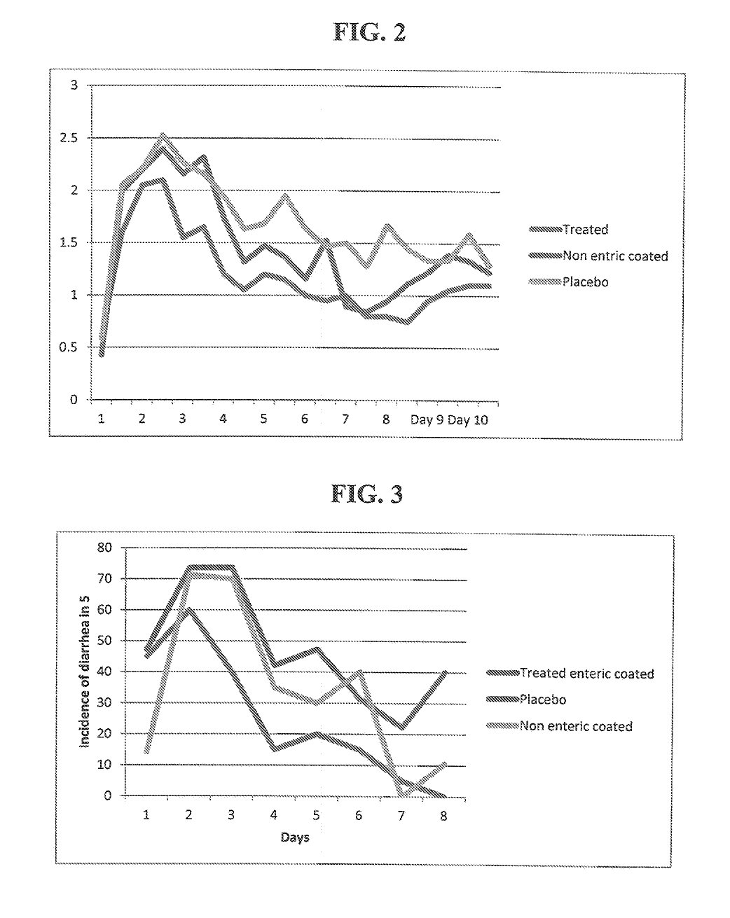

The results of this study showed a difference in response between placebo treated calves and those treated with SB 300. In addition, a difference in the response was observed between calves administered the enteric form of SB 300 and those administered the non-enteric, dissolved powder form, as observed before the end of treatment, as presented in FIG. 2. FIG. 2 shows that the difference between the average from the group of calves treated with enteric coated tablets of SB 300 and the average from the group of calves treated with the placebo was statistically significant before the end of treatment. (Day 4 AM, p<0.002, ANOVA). Thus, the calves treated with enteric SB 300 exhibited a significant improvement in fecal consistency over time of treatment.

With respect to animal morbidity, the percentage of calves having watery diarrhea (score of 2 and above) are presented in FIG. 3. Each calf was scored twice daily. The difference between the calves in the group treated with the enteric tablet form of SB 300 and the calves in the group treated with placebo is statistically significant on day 4 AM (p<0.05 Fischer test). Thus, the enteric SB 300 treated calves showed a better response (improvement in the diarrhea condition; fewer calves having watery diarrhea) than did those receiving the non-enteric form at the time of evaluation. (See, FIG. 3). Improvements in the average duration of watery diarrhea (fecal score of ≧2) and in the average duration of severe watery diarrhea (fecal score of 3 or 4) in the enteric SB 300 treated calves treated compared with the placebo treated calves are further observed in the below Table 3.

TABLE 3

| | Avg. Duration of | Avg. Duration of |
|---|---|---|
| | Watery Diarrhea | Severe Watery |
| | (Score ≧2) | Diarrhea (Score ≧2) |
| SB 300 enteric | 3.03 days | 1.10 days |
| coated tablets | | |
| Placebo | 5.16 days | 2.42 days |

In addition, the calves treated with enteric form of the SB 300 Croton lechleri proanthocyanidin polymer composition showed a greater average weight gain during the 25 day observation period compared with animals treated with a reconstituted powder form of SB 300 or placebo. (Table 4). This was an unexpected finding, especially because the pH in the calf stomach is relatively high, e.g., it can reach ˜pH 6.0 at the time of feeding, compared with a low, acidic pH in the stomachs of other animal species. In view of the high, less acidic pH in the calf stomach when treated with the SB 300 Croton lechleri proanthocyanidin polymer composition at the time of feeding with milk, it was unexpected that an enteric coated formulation would provide a treatment benefit for the calves, or would yield an improvement in the animal's diarrhea condition, relative to a non-enteric coated form of the composition. The finding that an enteric coated Croton lechleri proanthocyanidin polymer composition (SB 300) provided a better response in the young calves following its administration was a surprising and beneficial discovery related to the study.

An analysis of calves monitored during the progression of the study described in this example showed the average weight gain of animals (in pounds (lbs.)) between day 1 and day 25, as presented in Table 4. Animal mortality was also monitored during the 25 day study period. While deaths were seen among the groups of calves treated with either SB 300 powder or placebo; only one death occurred in the calf group treated with SB 300 in enteric coated tablet form.

TABLE 4

| | | Mortality (%)/ | Average weight |
|---|---|---|---|
| | | (Number of | gain after 25 |
| Treatment | Number of calves | deaths) | days (lbs.) |
| SB 300 enteric | 20 | 5% (1) | 15.5 lbs. |
| coated tablets | | | (281 g/day) |
| SB 300 powder | 21 | 23.8% (5) | 11.12 |
| Placebo | 19 | 21% (4) | 12.1 lbs. |
| | | | (219 g/day) |

A preliminary analysis of the above results showed the percent mortality of animals treated with placebo to be 21.5%, which is very close to the value determined at completion of the study and presented in Table 4; the number of animal deaths in this group was the same. In the preliminary analysis, the average weight gain after 25 days for animals treated with SB 300 enteric coated tablets was determined to be 11.94 lbs., and for animals treated with placebo, the average weight gain after 25 days was determined to be 9.30 lbs. Thus, the preliminary values and results are highly consistent with those determined at completion of the study, as presented in Table 4. The results from this study demonstrate a clear benefit to the use of SB 300 in enteric form to prevent calf mortality in animals sickened by diarrhea induced by infection with E. coli. In addition, the calves gained more weight when treated with enteric tablets of SB 300. A reduction in mortality and weight gain in animals treated with an SB 300 Croton lechleri proanthocyanidin polymer composition, particularly, the enteric form of SB 300, elucidates the advantages of the treatment methods of the invention. Increased weight of the study animals allowed the animals to become healthier and stronger during their treatment with the SB 300 Croton lechleri proanthocyanidin polymer composition. Decreasing animal morbidity, increasing weight gain and producing healthier animals with a reduced number of treatments all impact the health, nutrition and overall quality of animals in animal-focused industries worldwide. This further translates into commercial, financial and economic advantages and benefits to those in the calf production industry.

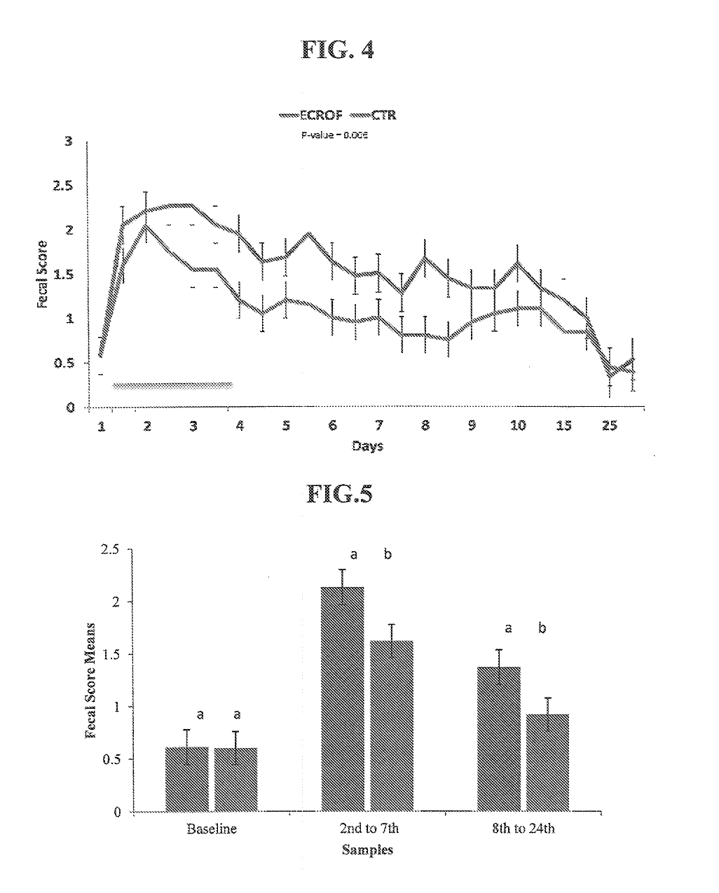

A significant result of the trial described in this Example was observed in connection with the fecal scores and fecal dry matter content of the treated calves of Group 1. A multivariate analysis of variance (MANOVA) of the results revealed a significant difference in the fecal scores between the treatment groups during the first 10 days of life. See, e.g., FIG. 4, 20 consecutive samples; p-value=0.018). As can be observed from FIG. 4, calves in the group treated with enteric coated C. lechleri botanical extract containing proanthocyanidin (ECROF group) had significantly lower fecal scores when compared to calves in the control (CTR) group. As observed from FIG. 5, pre-challenged fecal scores were not significantly different between treatment groups; 0.61 (SEM 0.16), 0.6 (0.15) for CTR, ECROF, respectively (P-value=0.90). Calves in the ECROF group (1.61±0.15) had significantly lower fecal scores during treatment when compared to control group (2.13±0.16, p-value=0.018). After treatment cessation, fecal scores were significantly lower for calves in the ECROF group (P-value=0.012) when compared to control calves.

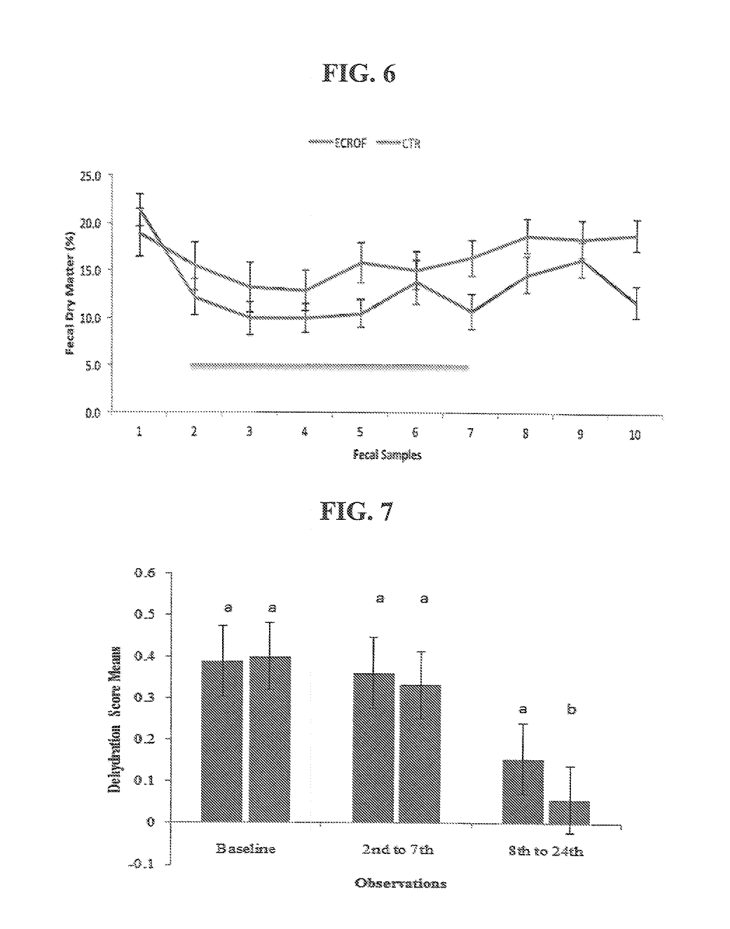

The fecal scoring data of FIGS. 4 and 5 were confirmed by the data reflecting the dry matter in the feces (See, e.g., FIG. 6). In FIG. 6, calves in the ECROF group had significantly higher fecal dry matter content when compared to calves in CTR (p-value=0.03). The results demonstrated treatment of animals with enteric coated C. lechleri botanical extract containing proanthocyanidin (e.g., SB 300) significantly decreased the severity of watery diarrhea and improved fecal scores over the course of a 25 day treatment period (p=0.0133).

In addition, no difference was observed in dehydration score between treatment groups before challenge (P-value=0.80) nor during treatment days (FIG. 7, p-value=0.67). However, after treatment cessation, lower dehydration scores were observed for the calf group treated with ECROF (P-value=0.03) compared to the dehydration scores in the control group.

A surprising and unexpected discovery resulting from this trial is that the effect of the administration of the C lechleri proanthocyandin extract containing product on diarrhea and dehydration lasted much longer than the period of time the product was given to the calves. While it was previously believed that the active component of the Croton lechleri derived product does not have a carryover effect, the results obtained from the clinical study and presented here surprisingly demonstrate that early administration of the product may induce beneficial changes in the intestines of the ECROF-treated animals that outlived the course of therapy. For example, administration of the product could favor the development of a healthier GI tract microbiota. In particular, early administration of a Croton lechleri derived proanthocyanidin product to an animal that presents with, is suspected of having, or is at risk of contracting diarrhea from various causes, such as infection or environmental factors, may offer long term protection to the animal, such as more than one, two, or three weeks, beyond the time of the last administration of the product to the animal.

### Example 4

### Administration of a Paste Containing SB 300 Enteric Beads to Treat Diarrhea in Equine Foals

This Example describes a study designed in accordance with the present invention to investigate the safety of a paste formulation of SB 300 on young horses (foals) as an antisecretory product for the treatment of diarrheal disease. The length of the entire study from acclimation to conclusion and final observations and sample collections was 2 weeks.

Diarrhea in young equines is very common and there are many causative agents and conditions (e.g., viral, bacterial, protozoa, drugs and toxins) that manifest clinical signs of watery diarrhea (secretory) in the foals. These agents and conditions trigger the pathophysiological mechanisms of secretory diarrhea caused by abnormal ion transport in intestinal epithelial cells. The presence of such abnormal mediators result in changes in intracellular cyclic adenosine monophosphate (cAMP), cyclic guanosine monophosphate (cGMP) calcium and/or protein kinases, which, in turn, cause an increase in chloride secretion. Water follows the chloride ions. Secretory diarrhea in afflicted animals is due to abnormal stimulation of the intestinal epithelium with a concomitant increase in the rate of intestinal secretion that overwhelms the absorptive capacity. Symptomatic treatment includes administration of anthelmintics, fluids, colloids, anti-inflammatory/analgesic, bismuth subsalicylate, probiotics and dietary modifications, with the principal goal to restore and maintain fluid and electrolyte balance. The use of antimicrobials in these cases is optimally restricted for foals with confirmed sepsis or a high likelihood of sepsis.

### Study Animals and Procedures

Thirty (30) foals on a breeding farm in Oklahoma were acclimated for seven days. Twenty four (24) foals were chosen for participation in the study. Six foals were dismissed from the study. The reasons for dismissal were as follows: one foal had an injured hind leg; two were lame; one had primary heart block when the heart was auscultated; and the two heaviest foals were removed. The heaviest foals were 75 pounds heavier than the heaviest foal included in the study. The lightest foal was 35 pounds less than the next lightest foal. The age range at the start of the study was four (4) months to ten (10) months. There were nine females and fifteen males. The breeds represented were twenty-one (21) quarter horse, 1 thoroughbred, 1 thoroughbred cross and 1 warm blood. While animals that are all of same breed are preferred, females, males, male castrates, or any breed were acceptable. Feeding and housing of the horses was in accordance with the standard of care at the study site.

The study was a blinded, randomized, controlled study conducted by the study veterinarian using animals residing on the contract location. Personnel administering treatments did not perform any assessments.

A 4 block randomization with a block size of 6 was utilized. All 24 foals were ranked from lowest to highest body weight. Then, using the RAND function of Excel, a random number was assigned to each foal. Using the random number, the foals were sorted into 4 treatment groups starting with the lowest random number.

The foals were stalled individually at night for the assessments and treatments and then turned out in compatible groups. Foals were feed concentrate ‘Nutrena Safe Choice Mare and Foal’ in stalls and had access to alfalfa hay (sourced from Colorado and Nebraska) ad libitium in the stall and in pastures. Water supply source was well water on the farm.

### Physical Examinations and Laboratory Testing

Physical exams and clinical laboratory testing were performed on Days −1 and 7 and included rectal temperature/heart rate/respiratory rate, mucous membrane color and hydration (CRT), activity/attitude, body weight indirect by weigh tape and body condition score using the Henneke Body Condition Scoring System.

Laboratory tests included blood drawn from all groups for complete blood count and chemistry profile and feces collected from the placebo and 5× groups for fecal culture (including screening for pathogenic E. coli; E. coli serotyping; Salmonella sp/Shigella sp.), Rota virus antigen, Clostridium perfringens enterotoxin assay and ova parasite egg counts.

### Treatments and Assessments

A paste formulation containing SB 300 was administered at a dose of 2 mg/kg. The treatment form was one tube containing 15 ml of the paste containing 880 mg of active SB 300, 58 mg/ml, for oral administration in equines only. For horses under 1 year of age, the dose was 1 ml/60 lbs. body weight. The formulation for administering to the foals in this Example was enterically coated (EC) SB 300 in the paste formulation; total of 880 mg of active (SB 300) per syringe, which is the equivalent of 1460 mg of SB 300 (880 mg active/0.6). The SB 300 lot used to make the EC beads contained approximately 60% active. Beads were approximately 45% SB 300; therefore, each syringe contained approximately 3.2 g EC SB 300 beads (1.460/0.45).

A representative paste composition of the present invention, which comprises enterically coated SB 300 beads, exemplifies the paste used in the study. For the purposes of administration to the animals in the study, the enteric SB 300 paste composition was contained in a syringe. The paste containing enteric coated SB 300 beads for use in the study contains the following components:

| Component | % w/w | Theoretical mg/syringe |
|---|---|---|
| SB 300 enteric beads | 21.91 | 3286.6* |
| Vegetable oil | 64.42 | 9663.5 |
| Cetyl alcohol | 9.76 | 1464.2 |
| Apple flavor | 0.08 | 11.7 |
| Silicon dioxide | 2.73 | 410.0 |
| Butylated hydroxytoluene | 0.04 | 5.9 |
| Titanium dioxide | 1.05 | 158.1 |
| Total | 100.0 | 15000 |

*3286.6 mg SB 300 enteric beads corresponds to 880 mg theoretical SB 300.

The foals were observed daily for 7 days prior to treatments for baseline assessments. The foals were treated twice a day, approximately 12 hours apart, for 3 days with either placebo (3× volume) or 1×, 3×, or 5×SP 300 (crofelemer) at the 1× dose of 2 mg/kg Body Weight. The time of the AM treatments was recorded. On Days 0, 1 and 2, the foals were assessed at 1, 4 had 8 hours post treatment, either in the stalls or in the paddocks. On Days 3 to 6, the foals were assessed twice a day at least four hours between observations. The evaluator (veterinarian or assistant) conducted the following assessments: fecal score, general attitude, activity, gastro-intestinal status and appetite. With respect to the 3× dose of SB 300, the 3× group received 3× placebo paste on Day 2 of the study. The supply of active paste became limited and on Day 2, only the 1× and the 5× groups were treated with active SB 300.

| Body | | | | | |
|---|---|---|---|---|---|
| Weight | Body Weight | Total mg 1X | | | |
| (BW) lbs. | (BW) lbs. | Dose* at | 1X ml | | 5X ml |
| Range | Mean | 2 mg/kg | dose | 3X ml dose | dose |
| 300-440 | 385 | 350 | 6 | 18 | 30 |
| 441-550 | 495 | 450 | 8 | 23 | 39 |
| 551-660 | 605 | 550 | 9 | 28 | 47 |
| 661-770 | 715 | 650 | 11 | 34 | 56 |
| 771-880 | 825 | 750 | 13 | 39 | 65 |

*Dose based on mean body weight
**Concentration for 1X, 3X and 5X ml doses: 58 mg/ml.

The evaluator (veterinarian or assistant) conducted assessments to determine a fecal score at baseline and on study Days −1-7 based on the parameters below:

| Fecal Description | Score |
|---|---|
| Well-formed stools with slightly moist surface which leave marks | 1 |
| when picked up | |
| Soft or very soft, moist, amorphous | 2 |
| Viscous liquid with some particulate matter | 3 |
| Watery, liquid stools with little particulate matter | 4 |
| Severe watery diarrhea, no particulate matter visible | 5 |
| Hemorrhagic diarrhea | 6 |
| None observed | 7 |

The foals' general attitude, activity and appetite were scored according to the parameters below, at baseline once a day during acclimation and on treatment days 0, 1, 2, at 1 hour+/−10 minutes, 4 hours+/−20 minutes and 8 hours+/−30 minutes post am treatment. Thereafter, these observations were recorded twice a day at least 4 hours apart on days 3 to 7.

| Attitude | Score |
|---|---|
| Normal | 0 |
| Slightly depressed | 1 |
| Moderately depressed | 2 |
| Severely depressed, animal not responsive to stimuli | 3 |

| Activity | Score |
|---|---|
| Normal | 0 |
| Slightly decreased (less interested in walking around) | 1 |
| Moderately decreased (resists walking around) | 2 |
| Severely decreased (recumbent and refuses to walk around) | 3 |

| Appetite | Score |
|---|---|
| Normal-Active nursing/creep feeding/eating hay | 0 |
| Ate most-Nurse intermittent/some creep and hay eating | 1 |
| Ate little-Little nursing or creep and hay feeding | 2 |
| Did not eat | 3 |

| Gastro-intestinal evaluation | Score |
|---|---|
| Normal | 0 |
| Slightly uncomfortable (stretching, looking at side for more than 5 | 1 |
| seconds) | |
| Moderately uncomfortable (rolling) | 2 |
| Severely uncomfortable (recumbent and refuses to walk around) | 3 |

### Evaluation of Safety

The safety of the treatments was measured by comparison of clinical assessments and clinical laboratory before and after treatments; by comparison of clinical assessments between control and treated groups; and by comparison of clinical laboratory tests between control and treated groups.

A physical examination/clinical evaluation was performed on Days −1 and 7, including assessment of Temperature/Heart rate/Respiratory rate; Mucous membrane color and Hydration (CRT) Activity/attitude; Body weight indirect by weigh tape; and Body condition score. Blood was drawn from all groups for the following: 1) Antec Diagnostics test L050 [CBC/CHEM PROFILE] prior to treatment (Day 1) and on last day of observations (Day 7) on foals in all groups. Feces were collected from the Control and 5× group horses (by rectal examination or from individual stall housing) prior to treatment (Day −1) and on last day of post treatment observations (Day 7); 2) Antec Diagnostics test M200 COMPLETE STOOL WORK UP evaluated for Fecal culture (including screening for pathogenic E. coli, E. coli serotyping; Salmonella sp/Shigella sp.), Rota virus antigen, Clostridium perfringens enterotoxin assay (2-5 grams feces and a Copan swab); 3) Antec Diagnostics test T805 OVA PARASITE [FEC], (2-5 grams feces).

Any animal suffering from unrelated problems (injuries, etc.) during the course of the study was withdrawn from the study. A necropsy was performed if a horse died. Data recorded up to the time of withdrawal or completion of the study, whichever is shorter, was used in the data analysis for the study.

### Statistical Methods

Statistical analyses were performed using commercial statistical software. Statistical differences was determined at the 5% level of significance using intention to treat analysis. Data were analyzed by analysis of variance (ANOVA).

For the statistical analysis of the clinical scores, a multivariate analysis of variance (MANOVA) with repeated-measures over time was used. Scores were assessed twice daily and a daily average was calculated and used as the outcome variable for the MANOVA model. Scores were recorded for all the foals twice daily for attitude, activity, attitude, appetite and GI evaluation daily for 7 days.

An ANOVA was used to compare foals enrolled in each treatment group prior to administration of the first treatment with respect to the mean of fecal scoring and all parameters (clinical chemistry and blood counts) tested from blood samples. An ANOVA was used to compare foals enrolled in each treatment group at exit from the trial with respect to the mean of fecal scoring and all parameters tested from the blood sample. An ANOVA is also used to compare daily weight gain (g) between treatment groups. The mean daily weight gain was calculated as the difference between exit and enrollment weight divided by the number of days a foal was part of the study trial.

### Results

The comparison of assessments of all foals in all groups, including placebo and treated, showed no changes in fecal score, appetite, activity, general attitude or gastro-intestinal status from the pretreatment period, treatment days, or post treatment days. All foals maintained normal activity, appetite and showed no signs of any gastro intestinal disturbances. One foal in the 5× treatment group had soft feces on Day −2, but the feces were normal for the rest of the study. One foal in the 3× treatment group had a onetime assessment of soft feces on Day 1 at 1 hour post treatment; and one foal in the 5× treatment group had a onetime assessment of soft feces on Day 6 at the AM assessment time. On Day 7 during the physical exam procedure, one foal in the 5× treatment group was reluctant to enter stocks; when it did, it pushed up against the front door and side panel and squatted on its hind legs. The foal then fell in stocks and froze with tetanic like head movements. The side panel was opened and the foal got up immediately. The foal was down for 20 seconds. The physical exam assessment showed that this foal was bright and alert, but its pupils were dilated with a reduced menace reflex. After 2 minutes, the menace reflex was re-checked and the animal was hyperactive. This was most likely due to the foal being frightened. This foal was normal throughout the days of pre-treatment, treatment, and post-treatment. When the foal was rechecked back in its stall 45 minutes later, it was normal.

All foals gained weight and maintained or increased the pre-treatment body score during the week following the treatments. The average weight gain for the placebo was 49 lbs.; the average weight gain for the 1× treatment group was 40 lbs.; the average weight gain for the 3× treatment group was 46 lbs.; and the average weight gain for the 5× treatment group was 60 lbs.

Fecal tests on the placebo and 5× treatment groups before and after treatments showed mixed gastrointestinal flora with no signs of any pathogenic organisms or toxins (E. coli, E. coli serotyping; Salmonella sp/Shigella sp., Rota virus antigen, Clostridium perfringens enterotoxin). Five out of six foals from of the placebo group and three of the foals in the 5× treatment group (2 foals did not have the test conducted as requested) had some evidence of intestinal parasites including strongyles, Parascaris equorum and anoplocephala before and these parasites were evident in five out of six foals from of the placebo and 5× groups after treatments.

Statistical analysis of the results from each group compared between Day −1 and Day 7 showed no differences for any CBC or CS parameters, except for GGT in the 1× group (p=0.037.) Because the 3× group and the 5× group showed no statistical differences, the difference in the 1× group is attributed to biological variation, especially because the values are all within normal ranges. For the Basophil analysis on Day 7, all values were 0 for the placebos; thus, although a statistical difference was shown (p=0.001), it was not meaningful with respect to the treatment groups. There were no early withdrawals of any foals or adverse events reported during the study. Based on the study data, it was concluded that the treatment product is safe to use in foals for treating diarrhea in accordance with the described methods.

All patents, patent applications and publications referred to or cited herein are hereby incorporated by reference in their entireties for all purposes.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

What is claimed is:

1. A method of treating a neonatal or young non-human animal having diarrhea associated with enteropathogenic infection, the method comprising orally administering to the neonatal or young non-human animal in need thereof a pharmaceutical composition comprising an aqueous soluble proanthocyanidin polymer from Croton lechleri, wherein the composition is formulated as a bolus or as a reconstituted powder and administered to the neonatal or young non-human animal in an amount of at least 40 mg to 300 mg per day for two or more consecutive days.

2. The method according to claim 1, wherein the neonatal or young non-human animal is selected from a bovine calf, a camel calf, a buffalo calf, a bison calf, a lamb, a kid, a foal, or a piglet.

3. The method according to claim 2, wherein the neonatal or young non-human animal is a bovine calf.

4. The method according to claim 2, wherein the neonatal or young non-human animal is a camel calf.

5. The method according to any one of claims 1 to 4, wherein the non-human animal is unweaned.

6. The method according to any one of claims 1 to 5, wherein the proanthocyanidin polymer composition is administered twice daily for three consecutive days.

7. The method according to any one of claims 1 to 6, wherein the enteropathogenic infection is with one or more of E. coli, rotavirus, or coronavirus.

8. The method according to claim 7, wherein the enteropathogenic infection is additionally with Salmonella spp. and/or Cryptosporidia.

9. The method according to any one of claims 1 to 8, wherein the proanthocyanidin polymer composition is administered as a powder reconstituted with oral electrolytes, milk or a milk substitute, physiological saline, or water.

10. The method according to any one of claims 1 to 9, wherein the proanthocyanidin polymer composition is administered as a bolus.

11. The method according to any one of claims 1 to 8, wherein the proanthocyanidin polymer composition is administered in animal feed.

12. The method according to any one of claims 1 to 11, wherein the non-human animal is less than two weeks of age.

13. The method according to any one of claims 1 to 11, wherein the non-human animal is two to four weeks of age.

14. The method according to any one of claims 1 to 13, wherein the composition is administered to the neonatal or young non-human animal in an amount of at least 30 mg to 350 mg.

15. The method according to any one of claims 1 to 14, wherein the composition is administered to the neonatal or young non-human animal in an amount of 250 mg.

16. The method according to any one of claims 1 to 13, wherein the neonatal or young non-human animal is a lamb or a kid, and the composition is administered in an amount of 40 mg or 50 mg.

17. The method according to any one of claims 1 to 16, wherein the neonatal or young non-human animal is approximately 30 to 50 kg in weight.

18. The method according to claim 3, wherein the bovine calf is approximately 30 to 40 kg in weight.

19. The method according to claim 4, wherein the camel calf is approximately 40 to 50 kg in weight.

20. The method according to any one of claims 1 to 19, wherein the proanthocyanidin polymer is administered as an enteric coated pharmaceutical composition.

21. The method according to any one of claims 1 to 19, wherein the proanthocyanidin polymer is administered as a non-enteric coated pharmaceutical composition.

22. The method according to any one of claims 1 to 21, wherein the proanthocyanidin polymer is selected from the group consisting of SB 300, SP 303, and crofelemer.

23. A method of improving weight gain or reducing mortality in neonatal non-human animal, said method comprising administering to said neonatal non-human animal a pharmaceutical composition comprising an aqueous soluble proanthocyanidin polymer from Croton lechleri, wherein the composition is formulated as a bolus or as a reconstituted powder and administered to the neonatal or young non-human animal in an amount effective to improve weight gain or reduce mortality for at least one day between the first and fourth days after birth of said neonatal non-human animal.

24. The method according to claim 23, wherein the neonatal non-human animal is selected from a bovine calf, a camel calf, a buffalo calf, a bison calf, a lamb, a kid, a foal, or a piglet.

25. The method according to claim 24, wherein the neonatal non-human animal is a bovine calf.

26. The method according to claim 24, wherein the neonatal non-human animal is a camel calf.

27. The method according to any one of claims 23 to 26, wherein the proanthocyanidin polymer composition is administered for at least three consecutive days.

28. The method according to any one of claims 23 to 27, wherein the proanthocyanidin polymer composition is administered as a powder reconstituted with oral electrolytes, milk or a milk substitute, physiological saline, or water.

29. The method according to any one of claims 23 to 28, wherein the composition is administered to the neonatal non-human animal in an amount of between 30 mg to 350 mg.

30. The method according to any one of claims 23 to 28, wherein the composition is administered to the neonatal animal in an amount of 250 mg.

31. The method according to any one of claims 23 to 30, wherein the proanthocyanidin polymer is administered as an enteric coated pharmaceutical composition.

32. The method according to any one of claims 23 to 30, wherein the proanthocyanidin polymer is administered as a non-enteric coated pharmaceutical composition.

33. The method according to any one of claims 23 to 32, wherein the proanthocyanidin polymer is selected from the group consisting of SB 300, SP 303, and crofelemer.

34. A method of treating a neonatal or unweaned equine animal for diarrhea associated with enteropathogenic infection, the method comprising orally administering to the animal a pharmaceutical composition comprising an aqueous soluble proanthocyanidin polymer from Croton lechleri, wherein the composition is provided in a form selected from a bolus, a reconstituted powder, or a gel, and is administered to the animal in an amount of at least 100 mg for two or more consecutive days.

35. The method according to claim 34, wherein the animal is infected with bacteria, viruses and protozoa, which infection induced the diarrhea.

36. The method according to claim 34 or 35, wherein the proanthocyanidin polymer composition is administered to the animal in an amount of at least 250 mg.

37. The method according to any one of claims 34 to 36, wherein the proanthocyanidin polymer composition is in the form of a gel contained in a delivery device.

38. The method according to claim 37, wherein the delivery device is a syringe.

39. The method according to claim 37 or claim 38, wherein the gel comprises polymeric microparticles or nanoparticles containing the composition.

40. The method according to claim 39, wherein the polymeric microparticles or nanoparticles are pH-sensitive.

41. The method according to any one of claims 34 to 40, wherein the animal is less than two weeks of age.

42. The method according to any one of claims 34 to 41, wherein the animal is approximately 30 to 50 kg in weight.

43. The method according to any one of claims 34 to 42, wherein the proanthocyanidin polymer is administered as an enteric coated pharmaceutical composition.

44. The method according to any one of claims 34 to 42, wherein the proanthocyanidin polymer is administered as a non-enteric coated pharmaceutical composition.

45. The method according to any one of claims 34 to 44, wherein the proanthocyanidin polymer is selected from the group consisting of SB 300, SP 303, and crofelemer.

46. A method of treating or preventing diarrhea in a neonatal or young equine foal, the method comprising orally administering to the foal in need thereof a pharmaceutical composition comprising an aqueous soluble proanthocyanidin polymer from Croton lechleri, wherein the composition is formulated as a paste and is orally administered to the foal in an amount of 2 mg/kg per day two times per day for two or more consecutive days.

47. The method according to claim 46, wherein the composition is formulated as a paste and is orally administered to the foal in an amount of 2 mg/kg per day two times per day for three consecutive days.

48. The method according to claim 46 or claim 47, wherein the paste is administered two times a day, twelve hours apart, for three consecutive days.

49. The method according to any one of claims 46 to 48, wherein the proanthocyanidin polymer is selected from the group consisting of SB 300, SP 303, and crofelemer.

50. The method according to any one of claims 46 to 49, wherein the paste comprises beads comprising enterically coated SB 300.

51. The method according to any one of claims 46 to 50, wherein the paste is contained in a delivery device.

52. The method according to claim 51, wherein the delivery device is a syringe.

53. The method according to any one of claims 46 to 52, wherein the oral administration comprises applying the paste to the roof of the foal's mouth.

54. The method according to any one of claims 1 to 53, wherein the C. lechleri proanthocyanidin polymer or composition thereof is administered in conjunction with probiotics.
